(19) United States
(12) Patent Application Publication
(10) Pub. No.: US 2026/0306765 A1
(43) Pub. Date: Oct. 8, 2026

(54) MULTIMODE NASAL MEDICATION INJECTOR AND METHODS RELATED THERETO

(71) Applicant: Flxion Medical, Inc., Magnolia, TX (US)

(72) Inventor: Thor Rollings Halseth, Magnolia, TX (US)

(21) Appl. No.: 19/465,454

(22) Filed: Jan. 30, 2026

Related U.S. Application Data
(60) Provisional application No. 63/783,113, filed on Apr. 3, 2025.
(60) Provisional application No. 63/905,050, filed on Oct. 24, 2025.

(51) Int. Cl. A61M 15/08; A61M 5/28; A61M 5/31; A61M 5/315

(52) U.S. Cl. CPC A61M 15/08; A61M 5/288; A61M 5/3134; A61M 5/31513

(57) ABSTRACT

Single injector, i.e., a multimode nasal injector, that can be used for intranasal, intramuscular, and intravenous medication delivery; medication syringes that isolate the needle from the medication in the syringe prior to medication delivery, which syringes may be used with the multimode nasal injector.

### RELATED APPLICATIONS

This application claims the benefit of priority of U.S. Provisional Application No. 63/783,113, filed on Apr. 3, 2025 and U.S. Provisional Application No. 63/905,050, filed on Oct. 24, 2025, the entire contents of which application(s) are incorporated herein by reference. To the extent that any material incorporated by reference conflicts with the express disclosure of this application, the present disclosure shall control and govern any such conflicting material, including any conflicting definitions of terms.

### FIELD OF THE DISCLOSURE

The present disclosure relates to a nasal medication injector, and more particularly to a nasal medication injector that may be converted for use as an intramuscular and/or intravenous medication injector; the present disclosure also relates to medication syringes, and more particularly to medication syringes that isolate the needle from the medication in the syringe prior to medication delivery; the syringes may be used with the multimode nasal injector of the present disclosure.

### BACKGROUND OF THE DISCLOSURE

According to the CDC there were an estimated 100,306 drug overdose deaths in the United States in 2021, an increase of 28.5% over the same period the year before. Fentanyl has become the leading cause of death for adults ages 18-45 in the United States. In the year ending in April 2021, CDC data shows that fentanyl killed 40,010 Americans between the ages 18 and 45, nearly twice as many as car accidents at 22,442 deaths, suicide 21,678 deaths, COVID 21,335 deaths, and cancer 17,114 deaths.

Recovery from an otherwise fatal opioid overdose or anaphylaxis involves the administration of naloxone or epinephrine using either intranasal (IN), intramuscular (IM), or intravenous (IV) medication delivery. For IM, current devices are often pre-filled, ready-to-use injectors, and there are many ready-to-use injectors on the market today. While easy to use, current injectors have many parts which include powerful, expensive springs to inject the medication into the thigh and may contain up to 26 parts; as such, these injectors are complicated to assemble, making them very expensive for the end user. Thus, there is a need in the art for an injector that is more affordable and thus more readily available to those who need them for survival from potentially deadly opioid or anaphylaxis episodes.

Further, while intranasal may be a preferred first option for medication delivery, if the nasal passages are blocked, such as by mucus, vomit or otherwise, it can be impossible to administer medication intranasally. Hence, authors of the present disclosure have recognized a need in the art for a single delivery device that includes multiple modalities beyond intranasal (intranasal, intramuscular, and intravenous) and which overcomes many of the limitations and drawbacks of existing nasal devices and separate auto-injector-type devices, especially in life threatening situation where seconds matter. The authors also recognize the need for a device that is affordable to those who depend on them for survival from an opioid or anaphylaxis episode.

### SUMMARY OF THE DISCLOSURE

Exemplary devices and methods of the present disclosure provide the user with a multimode nasal injector that has multiple medication delivery modalities in a single device: intranasal, intramuscular, and intravenous. In this regard, the multimode nasal injector of the present disclosure overcomes drawbacks and limitations of prior devices, especially in life threatening situations where seconds matter. Exemplary devices and methods of the present disclosure also provide medication syringes, in particular medication syringes that isolate the needle from the medication in the syringe prior to medication delivery; such syringes may be used with the multimode nasal injector of the present disclosure.

In a first, preferred configuration, as initially supplied to the user, the multimode nasal injector can be supplied with a nasal nozzle removably and fixedly attached to the injector in such a manner that the nozzle will not inadvertently become detached from the injector, such as by sliding off. As supplied, the multimode nasal injector is configured so that the user does not see and cannot touch the needle, reducing needle-phobia and potential for needle contamination. When used in the intranasal mode the user will never see the needle, since it will remain entirely enclosed in the injector throughout use. If, however, the nasal passages of the user are blocked and it is impossible to administer medication intranasally, the multimode nasal injector of the present disclosure can be converted from intranasal delivery to another modality, such as IM or IV, by simply removing the nasal nozzle. (Even for IM delivery the needle need not be seen during injection or after, because the injector is pressed against the user's tissue when the needle is extended, and the needle is automatically retracted after delivery of the medication even with the nozzle removed.)

Turning to exemplary structures in more detail, multimode nasal injectors of the present disclosure may include front and rear housings slidably coupled to one another along a longitudinal axis thereof. The front and rear housings define a housing cavity therein for holding a medication-containing needle-based injection system (NIS), such as a cartridge or syringe assembly. The medication include without limitation naloxone, epinephrine, ketamine or other medicaments, for example. A needle may be disposed in the cavity along the longitudinal axis for communication with the medication in the NIS. Desirably, the needle may be disposed in spaced apart relation out of fluid communication with the medication in the NIS prior to use. In this case, movement of a plunger seal will cause a medication seal in the NIS to be pierced by a proximal end of a double-ended needle to create fluid communication between the needle and the medication in the NIS. The plunger seal is capable of moving from an initial position closer to a proximal plunger end of the NIS to a second position closer to a distal needle end of the NIS, thereby causing the medication to be expelled from the NIS through the needle. Alternatively, the needle may be disposed in fluid communication with the medication in the NIS prior to use and may include a seal on a distal end of the needle to maintain a hermetic seal for the medication.

A plunger is connected to the plunger seal at a distal end thereof. A proximal, opposing end of the plunger may be provided as an integral part of the rear housing of the NIS, so the plunger and rear housing are seamlessly connected as a unitary whole when the plunger and rear housing move together during use of the multimode nasal injector. Alternatively, the plunger may be provided as a separately formed part which is used without the NIS or is permanently and fixedly attached to the rear housing of the NIS. Movement of the front and rear housings towards one another, or movement of the plunger without the NIS, will cause the plunger to move the plunger seal towards the distal needle end of the NIS, causing the egress of medication from the NIS through the needle. For intranasal medication delivery, the needle will extend into, and be fully enclosed in, the nasal nozzle, and the medication will be expelled from the needle and out from the injector through one or more openings in the nasal nozzle. The total range of travel of the front and rear housings may be limited by a medication spacer disposed around the plunger at the proximal end of the plunger in the NIS. The medication spacer has a thickness which limits the travel of the front and rear housings towards one another and thus limits the travel of the plunger seal and the amount of medication expelled from the multimode nasal injector.

If the medication is to be delivered intramuscularly or intravenously, the nasal nozzle is removed from the front housing of the multimode nasal injector prior to use, thus converting from a nasal injector to an IM/IV injector. With the nasal nozzle removed, when the front housing and rear housing are pressed together, the needle is extended forward out of the front housing at the distal end thereof. The medication is then ejected from the needle to the desired delivery location, IM or IV. In one desirable configuration, the needle extends 0.625 inches out of the front housing, which is particularly suited to IM injections. It should be noted that the length that the needle extends out of the front housing is determined by the choice of the size of the needle and other components and is not limited to just 0.625 inches.

After the medication is delivered, whether IN/IM/IV, and the user releases the front and rear housings, a biasing member disposed therein, such as a spring, which has been compressed by the user during medication delivery, will cause the front and rear housings to move away from each other into a final, lock-out position, where lockout structures on/in the front and rear housings cooperate to prevent the housings from moving relative to one another. In this final lock-out position the needle is again fully contained in the multimode nasal injector.

The multimode nasal injector of the present disclosure can be inexpensive to manufacture due to the relatively fewer parts and increased ease of assembly, thus being more affordable to those at risk of death or injury from an opioid or anaphylaxis episode. The multimode nasal injector of the present disclosure, with as few as 5 parts, can omit the powerful injection spring, seal, and other parts found in existing commercial injectors and can replace the force of the spring on the plunger with a motive force supplied by the user, i.e., the force of a physical motion of the user pushing the injector into the thigh or squeezing the front and rear injector housings together between the thumb and fingers. In this way, the invention eliminates the many parts and springs used in multiple devices currently on the market to accomplish the same end results with a single device.

For instance, an exemplary method for assembly of the multimode nasal injector described above may include the steps of: providing the front and rear housings, optional medication spacer, NIS, spring, needle, and injector cap; placing the optional medication spacer onto the plunger at a proximal end of the plunger; attaching a needle to the distal end of the NIS and inserting the NIS into the rear housing and placing the plunger seal in contact with the plunger of the rear housing (alternatively the NIS may be provided with the needle already attached); sliding a spring about the NIS and plunger with a proximal end of the spring in contact with a stop of the rear housing proximate the proximal end of the plunger; attaching the front housing over the NIS, needle and spring; fixedly and removably attaching the nasal nozzle to the distal end of the front housing, the nasal nozzle optionally having a self sealing membrane disposed therein and optionally engaging the membrane with a tip of the needle; and sliding a cap over the front housing and removably coupling the cap to the rear housing.

In a first desirable configuration the present disclosure provides a medication syringe comprising an elongated barrel having a barrel plunger end and an opposing barrel needle end and a longitudinal axis extending therebetween, the barrel having a cavity therein for holding a medication. A plunger seal is slidably disposed in the cavity proximate the barrel plunger end and a needle seal is disposed in the cavity proximate the barrel needle end, the plunger seal and needle seal defining a medication space therebetween in the cavity; the plunger seal and needle seal cooperate to retain a medication in the medication space. A double-ended needle is attached to the needle end of the elongated barrel with a selected end of the double-ended needle disposed in the cavity between the needle seal and the needle end of the barrel. The plunger seal is slidable within the cavity along the longitudinal axis and movement of the plunger seal towards the needle end of the barrel causes a medication to move towards the double-ended needle, causing the needle seal to move into contact with the selected end of the double-ended needle to pierced the selected end to place the double-ended needle in fluid communication with the medication space.

The needle seal may slidably disposed in the cavity and movement of the plunger seal towards the needle end of the barrel causes a medication to move towards the double-ended needle, causing the needle seal to slide into contact with the selected end of the double-ended needle to be pierced by the selected end to place the double-ended needle in fluid communication with the medication space. Additionally or alternatively, movement of the plunger seal towards the needle end of the barrel causes a medication to move towards the double-ended needle, causing a selected surface of the needle seal to expand outwardly towards the needle end of the barrel to be pierced by the selected end to place the double-ended needle in fluid communication with the medication space.

In another of its aspects, the present disclosure provides a medication syringe, comprising an elongated barrel having a barrel plunger end and an opposing barrel needle end and a longitudinal axis extending therebetween, the barrel having a cavity therein for holding a medication; a plunger seal is slidably disposed in the cavity proximate the barrel plunger end and a needle seal is disposed in the cavity proximate the barrel needle end, the plunger seal and needle seal defining a medication space therebetween in the cavity, the plunger seal and needle seal cooperating to retain a medication in the medication space. A double-ended needle is provided attached to a needle hub; the needle hub is attachable to the barrel needle end so that upon placement of the needle hub on the barrel needle end the medication seal is pierced by a selected end of the double-ended needle to place the double-ended needle in fluid communication with the cavity to receive medication therefrom. The needle hub may include a sidewall having a shape that is complementary to a shape of the barrel needle end, and the sidewall may have a plurality of longitudinal segments that extend along the direction of the longitudinal axis, the longitudinal segments configured to expand in an outward direction from the longitudinal axis to exert a gripping force on the barrel needle end when the sidewall is in contact therewith.

In a further of its aspects the present disclosure provides a medication syringe, comprising an elongated barrel having a barrel plunger end and an opposing barrel needle end and a longitudinal axis extending therebetween, the barrel having a cavity therein for holding a medication. A plunger seal is slidably disposed in the cavity proximate the barrel plunger end. A medication plug having a medication seal disposed therein is provide. The medication plug is attached to the needle end of the elongated barrel with the medication seal in contact with the needle end of the elongated barrel to prevent the egress of medication from the cavity through the barrel needle end. A double-ended needle is provided attached to a needle hub; the needle hub attachable to the medication plug so that upon placement of the needle hub on the needle plug the medication seal is pierced by a selected end of the double-ended needle to place the double-ended needle in fluid communication with the cavity to receive medication therefrom. The medication plug may be formed from a rigid material or may be formed from an elastic material, in which case the medication seal may be formed from the same material as the medication plug and the medication seal and the medication plug formed as a single unitary part.

In yet another of its aspects the present disclosure provides a multimode nasal medication injector housing for use with a needle-based injection system (NIS). The multimode nasal medication injector housing is capable of conversion from a nasal modality of medication delivery to an intravenous delivery mode and an intramuscular medication delivery mode. The multimode nasal medication injector housing includes a front housing having a front housing distal end and a front housing proximal end, and a rear housing having a rear housing distal end and a rear housing proximal end. The front housing proximal end slidably couples to the rear housing distal end to define a housing cavity therein and to permit the front and rear housings to translate relative to one another along a longitudinal axis extending therethrough. A plunger is permanently attached interior to the rear housing at a selected location at the rear housing proximal end, with the plunger extending along the longitudinal axis and a having opposing distal and proximal plunger ends, the proximal plunger end in contact with the rear housing. A nasal nozzle is provided having distal and proximal nozzle ends with a canal disposed interior thereto between the distal and proximal nozzle ends, the nasal nozzle extending along the longitudinal axis. The proximal nozzle end is removably coupled to the front housing at the front housing distal end. The nasal nozzle has one or more openings at the distal nozzle end through which the medication is delivered from the multimode nasal medication injector housing. The plunger may be an integral part of the rear housing so the plunger and rear housing are seamlessly connected as a unitary whole. A medication spacer may be provided having a thickness extending along the direction of the longitudinal axis. The medication spacer is disposed internal to the housing cavity at the selected location at the rear housing proximal end.

In still another of its aspects, the present disclosure provides a further multimode nasal medication injector housing for use with a needle-based injection system (NIS). The multimode nasal medication injector housing is capable of conversion from a nasal modality of medication delivery to an intravenous delivery mode and an intramuscular medication delivery mode. The multimode nasal medication injector housing includes a front housing having a front housing distal end and a front housing proximal end and a rear housing having a rear housing distal end and a rear housing proximal end. The front housing proximal end slidably couples to the rear housing distal end to define a housing cavity therein and to permit the front and rear housings to translate relative to one another along a longitudinal axis extending therethrough. A plunger is disposed in the housing cavity at a selected location at the rear housing proximal end, the plunger extending along the longitudinal axis and having a opposing distal and proximal plunger ends, the proximal plunger end in contact with the rear housing. A medication spacer may be provided having a thickness extending along the direction of the longitudinal axis, the medication spacer disposed internal to the housing cavity at the selected location at the rear housing proximal end. A nasal nozzle is provided having distal and proximal nozzle ends with a canal disposed interior thereto between the distal and proximal nozzle ends, the nasal nozzle extending along the longitudinal axis. The proximal nozzle end is removably coupled to the front housing at the front housing distal end. The nasal nozzle has one or more openings at the distal nozzle end through which the medication is delivered from the multimode nasal medication injector housing. The medication spacer may have an annular shape with a hole therein and may be disposed around the plunger with the plunger in the hole.

The plunger of any of the multimode nasal medication injector housings of the present disclosure may be configured to be attached to a plunger seal in an needle-based injection system. Exemplary housings of the present disclosure may also include a lockout structure comprising a tab disposed on a selected one of the first and second housings and having a complementary slot disposed on the other one of the first and second housings with the slot dimensioned to receive and hold the tab. The tab may be disposed on a proximal end of the front housing and the slot is disposed on a distal end of the rear housing. The rear housing may include a channel in which the tab can travel, upon relative movement between the front and rear housings. The tab and slot cooperate to lock the front and rear housings at a relative location that encloses the needle entirely within the injector when the tab is in the slot to prevent movement between the front and rear housings.

Any of the multimode nasal medication injector housings of the present disclosure may include a needle-based injection system (NIS) disposed in the housing cavity. The NIS contains the medication to be delivered, and has opposing proximal and distal NIS ends and disposed along the longitudinal axis. The NIS has a plunger seal slidably disposed therein operably connected to the distal plunger end of the housing, and has a needle disposed along the longitudinal axis between the distal NIS end and the front housing distal end. Housing of the present disclosure may include a biasing member, such as a spring. The spring may be positioned in the housing cavity along the longitudinal axis between the front and rear housings. The spring is configured to exert force outwardly to keep the front housing and the rear housing positioned in a first position prior to use. The spring is compressible upon application of force along the longitudinal axis to urge the front housing distal end towards the rear housing proximal end so that the front and rear housings are positioned at a second position and such that the needle is disposed in fluid communication with the medication in the NIS and the needle extends outward beyond the front housing distal end a selected length. The spring may have elastic potential energy stored therein when compressed in the second position. The elastic potential energy is sufficient to move the front and rear housings away from each other in a direction along the longitudinal axis to a third position upon release of the elastic potential energy. In the third position the needle is fully enclosed in the housing cavity of the front and rear housings. Desirably, the needle is not in fluid communication with the medication when the front and rear housings are in the first position. In this regard, the needle may be in fluid communication with the medication in the needle-based injection system when the front and rear housings are in the second position. When the injector housing is moved from the first position to the second position, the plunger seal pushes the medication out of the needle-based injection system, through the needle, and out of a distal end of the needle for delivery to a patient. A proximal end of the double-ended needle is configured to pierce a seal on the needle-based injection system upon movement of the front and rear housings towards one another thereby placing the double-ended needle in fluid communication with the medication in the needle-based injection system. The nasal nozzle may have a length greater than a the aforementioned selected length to contain the needle in the canal when the injector is in the second position. Further the NIS may be any of the exemplary medication syringes of the present disclosure. The spring may be disposed around the elongated barrel. The medication syringe may include a flange at the barrel plunger end; the spring may have two opposing ends and a selected end disposed against the flange. As an alternative to a medication syringe, the NIS may include a medication cartridge containing the medication to be delivered. In the case of the medication cartridge, the spring may be disposed around the plunger, and the spring has two opposing ends and a selected end may be disposed against the rear housing distal end.

In still yet a further of its aspects, the present disclosure provides method of assembling a multimode nasal medication injector capable of conversion from a nasal modality of medication delivery to an intravenous delivery mode and an intramuscular medication delivery mode. The method comprises providing a multimode nasal medication injector housing of the present disclosure; inserting a needle-based injection system (NIS) containing the medication to be delivered in the housing cavity along the longitudinal axis, the needle-based injection system having a plunger seal slidably disposed therein; operably connecting the distal plunger end of the plunger to the plunger seal of the NIS; placing a spring around the plunger with a proximal end of the spring in contact with a distal spring abutment surface, the spring configured to exert force outwardly to keep the front housing and the rear housing positioned in a first position prior to use, the spring being compressible upon application of force along the longitudinal axis to urge the front housing distal end towards the rear housing proximal end so that the front and rear housings are positioned at a second position and such that the needle is disposed in fluid communication with the medication in the needle-based injection system and the needle extends outward beyond the front housing distal end a selected length; attaching the front housing over the NIS; and fixedly and removably attaching the nasal nozzle to the front housing distal end. The NIS used in the method may include a medication syringe having opposing needle and plunger ends and includes a flange at the barrel plunger end and wherein the distal spring abutment surface is the flange. The NIS in the method may be any of the exemplary medication syringes of the present disclosure. Alternatively, the NIS in the method may include a medication cartridge and wherein the distal spring abutment surface a selected surface of the rear housing at the rear housing distal end. In a further aspect of the method, the method may include, prior to placing the spring, providing a medication spacer having a thickness that extends along the direction of the longitudinal axis and placing the medication spacer in the housing cavity at the selected location at the rear housing proximal end. The method may also include attaching a removable cap to the front housing distal end to cover the nasal nozzle.

### BRIEF DESCRIPTION OF THE DRAWINGS

The foregoing summary and the following detailed description of exemplary embodiments of the present disclosure may be further understood when read in conjunction with the appended drawings, in which:

FIG. 1A schematically illustrates a perspective view of an exemplary embodiment of a multimode nasal injector of the present disclosure;

FIG. 1B schematically illustrates a top view of the injector of FIG. 1A;

FIG. 2A schematically illustrates a top, longitudinal cross-sectional view of the injector of FIG. 1B taken along the sectioning lines 2A-2A;

FIG. 2B schematically illustrates the view of FIG. 2A but with the injector in a compressed configuration during medication delivery;

FIG. 2C schematically illustrates the view of FIG. 2A but with the injector returned to the position shown in FIG. 2A;

FIG. 3A schematically illustrates a top view of the injector of FIG. 1A with the cap on;

FIG. 3B schematically illustrates a top view of the injector of FIG. 3A with the cap off and in a first position for nasal use;

FIG. 3C schematically illustrates a top view of the injector of FIG. 3A with the cap off and in the compressed configuration during medication delivery for nasal use;

FIG. 3D schematically illustrates a top view of the injector of FIG. 3A with the cap off and returned to the first position after nasal use;

FIG. 4 schematically illustrates a perspective view of the injector shown in FIG. 3C;

FIG. 5A schematically illustrates a top view of an exemplary embodiment of the injector of FIG. 1A with the cap off and in a first position in preparation for intramuscular use;

FIG. 5B schematically illustrates a top view of the injector of FIG. 5A with both the cap and nasal nozzle off and in a first position for intramuscular use;

FIG. 5C schematically illustrates a top view of the injector of FIG. 5A in a compressed position disposed against patient tissue with the needle disposed in muscle tissue during medication delivery;

FIG. 5D schematically illustrates a top view of the injector of FIG. 5C returned to the first position after intramuscular use;

FIGS. 6A-6C schematically illustrate top, longitudinal cross-sectional views of the injectors shown in FIGS. 5B-5D, respectively;

FIG. 7A schematically illustrates a perspective view of the injector of FIG. 5B with the nasal nozzle off and in a first position for intramuscular use;

FIG. 7B schematically illustrates a top view of the injector of FIG. 7A;

FIG. 8A schematically illustrates a perspective view of the injector of FIG. 5C in the second position for intramuscular use, but without the patient tissue shown;

FIG. 8B schematically illustrates a top view of the injector shown in FIG. 5C, but with the user's hand in position on the injector;

FIGS. 9A-9C schematically illustrate top, longitudinal cross-sectional views of a multimode nasal injector of the present disclosure with lockout structure shown, which may include the injectors of FIGS. 1A-8B, in which FIG. 9A shows the injector in a first, initial position, FIG. 9B shows the injector in a second position during use, and FIG. 9C shows the injector in a third, lockout position after use;

FIGS. 10A-10F schematically illustrate top, longitudinal cross-sectional views of the multimode nasal injector of FIGS. 9A-9C showing the various respective configurations of the injector during use for intranasal delivery starting with the initial state with cap in place, FIG. 10A, and concluding with a view showing the injector in lockout position after use, FIG. 10F, with intermediates states shown in sequence between FIGS. 10A and 10F;

FIGS. 11A-11D schematically illustrate top views of multimode nasal injector of FIGS. 9A-9C during use starting in the initial state with cap in place, FIG. 11A, and concluding with a view showing the injector in lockout position after use, FIG. 11D;

FIGS. 12A-12E schematically illustrate top, longitudinal cross-sectional views of the multimode nasal injector of FIGS. 9A-9C showing the various respective configurations of the injector during use for intramuscular delivery starting with the initial state with cap in place, FIG. 12A, and concluding with a view showing the injector in lockout position after use, FIG. 12E, with intermediates states shown in sequence between FIGS. 12A and 12E;

FIGS. 13A-13E schematically illustrate top, longitudinal cross-sectional views of the multimode nasal injector of FIGS. 9A-9C showing the various respective configurations of the injector during use for intravenous use delivery starting with the initial state with cap in place, FIG. 13A, and concluding with a view showing the injector in lockout position after use, FIG. 13E, with intermediates states shown in sequence between FIGS. 13A and 13E;

FIGS. 14A-14D schematically illustrate top, longitudinal cross-sectional views of a multimode nasal injector having a medication spacer (which may include the injectors of FIGS. 1A-13E) and showing the various respective configurations of the injector during use for intramuscular delivery starting with the initial state with cap in place, FIG. 14A, and concluding with a view showing the injector in lockout position after use, FIG. 14D, with intermediates states shown in sequence therebetween;

FIG. 14E schematically illustrates a top, longitudinal cross-sectional view of an injector similar to that of FIGS. 14A-14D but having a relatively thinner medication spacer allowing for a greater amount of medication to be dispensed;

FIG. 15 schematically illustrates an exploded perspective view of the multimode nasal injector of FIGS. 9A-15E;

FIGS. 16A-20C schematically illustrate exemplary embodiments of medication syringes in accordance with the present invention in which a needle is isolated from the medication in the syringe prior to medication delivery;

FIGS. 16A-16C schematically illustrate a cross-sectional view of an exemplary medication syringe in which FIG. 16A shows a needle seal of the present invention, FIG. 16B shows the needle seal slidably disposed in a cavity of the syringe, isolating the needle from the medication, and FIG. 16C shows the needle seal slid onto and pierced by the needle so the needle is in communication with the medication during use;

FIGS. 17A-17C schematically illustrate a cross-sectional view of an exemplary medication syringe in which FIG. 17A shows a needle seal of the present invention, FIG. 17B shows the needle seal disposed in a cavity of the syringe, isolating the needle from the medication, and FIG. 17C shows the needle seal expanded and pierced and the needle in communication with the medication during use;

FIGS. 18A-18D schematically illustrate a cross-sectional view of an exemplary medication syringe including a double-ended needle attached to a needle hub, in which FIG. 18A shows a needle seal of the present invention, FIG. 18B shows the needle seal disposed in a cavity of the syringe, isolating the needle from the medication, and FIG. 18C shows the needle hub on the syringe with the needle seal pierced and the needle in communication with the medication during use, and FIG. 18D shows a cross-sectional and end view of the needle hub;

FIG. 19A schematically illustrates a cross-sectional, exploded view of an exemplary medication syringe of the present invention including a double-ended needle attached to a needle hub, and a medication plug having a needle seal disposed therein isolating the needle from the medication;

FIG. 19B schematically illustrates the syringe of FIG. 19A with the needle hub on the syringe prior to puncture of the medication seal;

FIG. 19C schematically illustrates the syringe of FIG. 19A with the needle hub on the medication plug after puncture of the medication seal, with the needle in communication with the medication;

FIG. 20A schematically illustrates a cross-sectional, exploded view of an exemplary medication syringe of the present invention including a double-ended needle attached to a needle hub, and a medication plug having a needle seal disposed therein isolating the needle from the medication with the medication plug and needle seal made from the same material as a single unitary part;

FIG. 20B schematically illustrates the syringe of FIG. 20A with the needle hub on the syringe prior to puncture of the medication seal;

FIG. 20C schematically illustrates the syringe of FIG. 20A with the needle hub on the medication plug after puncture of the medication seal, with the needle in communication with the medication;

FIGS. 21A-21C schematically illustrate top, longitudinal cross-sectional views of an exemplary multimode nasal injector housing of the present invention containing the syringe of FIGS. 16A-16C, showing the various respective configurations of the injector during use for intranasal delivery starting with the initial state, FIG. 21A, then the medication delivery state, FIG. 21B, and concluding with a view showing the injector in lockout position after use, FIG. 21C;

FIGS. 22A-22C schematically illustrate top, longitudinal cross-sectional views of an exemplary multimode nasal injector housing of the present invention containing the syringe of FIGS. 17A-17C, showing the various respective configurations of the injector during use for intranasal delivery starting with the initial state, FIG. 22A, then the medication delivery state, FIG. 22B, and concluding with a view showing the injector in lockout position after use, FIG. 22C;

FIGS. 23A-23C schematically illustrate top, longitudinal cross-sectional views of an exemplary multimode nasal injector housing of the present invention containing the syringe of FIGS. 18A-18C, showing the various respective configurations of the injector during use for intranasal delivery starting with the initial state, FIG. 23A, then the medication delivery state, FIG. 23B, and concluding with a view showing the injector in lockout position after use, FIG. 23C;

FIGS. 24A-24C schematically illustrate top, longitudinal cross-sectional views of an exemplary multimode nasal injector housing of the present invention containing the syringe of FIGS. 19A-19C, showing the various respective configurations of the injector during use for intranasal delivery starting with the initial state, FIG. 24A, then the medication delivery state, FIG. 24B, and concluding with a view showing the injector in lockout position after use, FIG. 24C; and

FIGS. 25A-25C schematically illustrate top, longitudinal cross-sectional views of an exemplary multimode nasal injector housing of the present invention containing the syringe of FIGS. 20A-20C, showing the various respective configurations of the injector during use for intranasal delivery starting with the initial state, FIG. 25A, then the medication delivery state, FIG. 25B, and concluding with a view showing the injector in lockout position after use, FIG. 25C.

### DETAILED DESCRIPTION

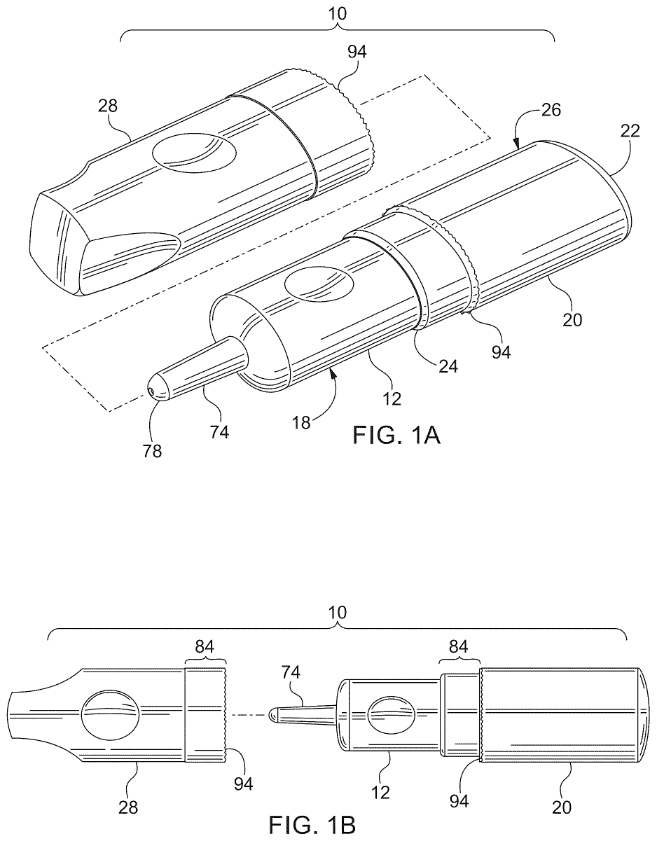

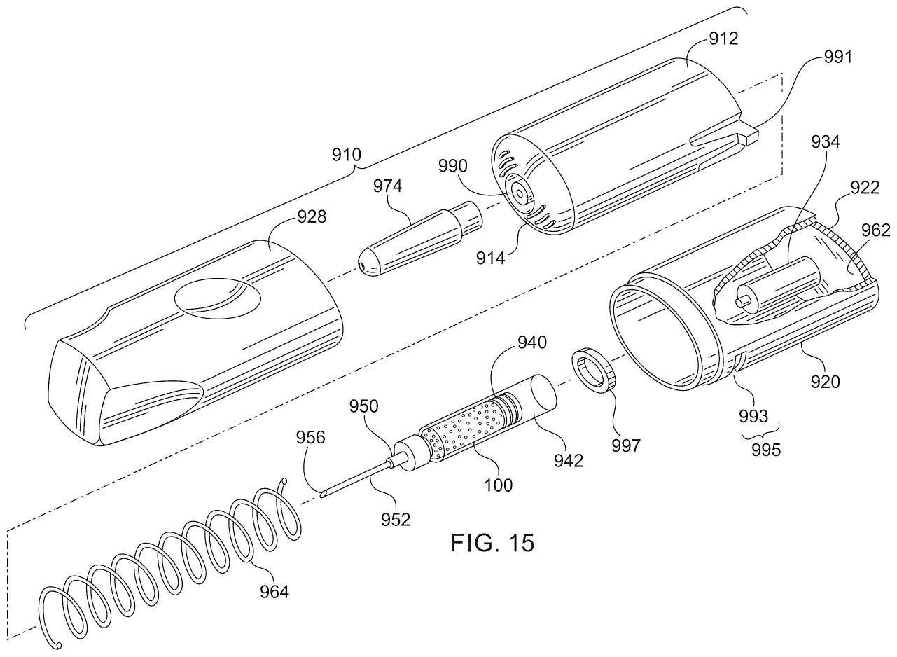

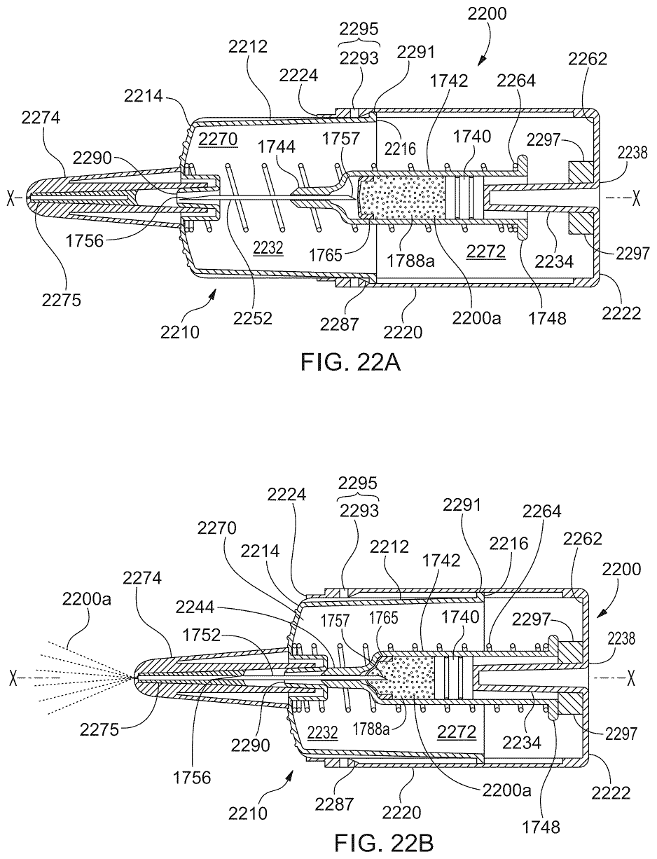

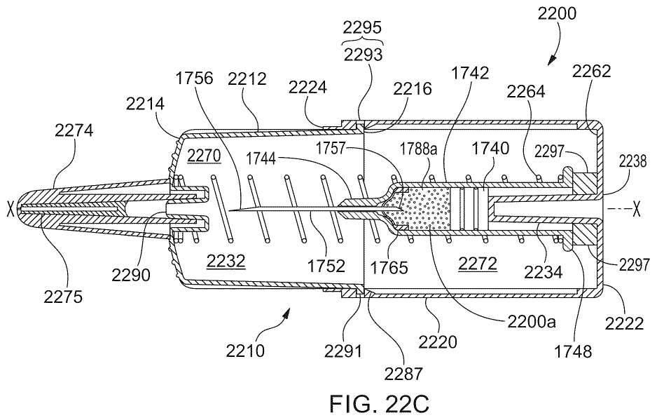

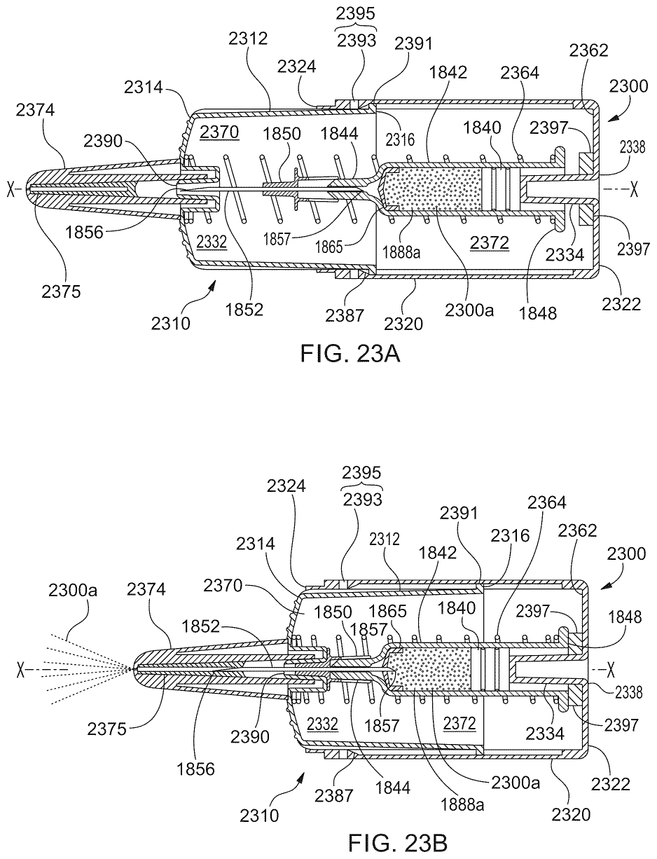

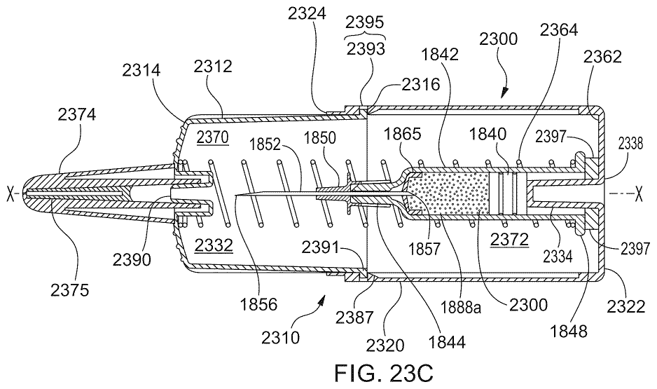

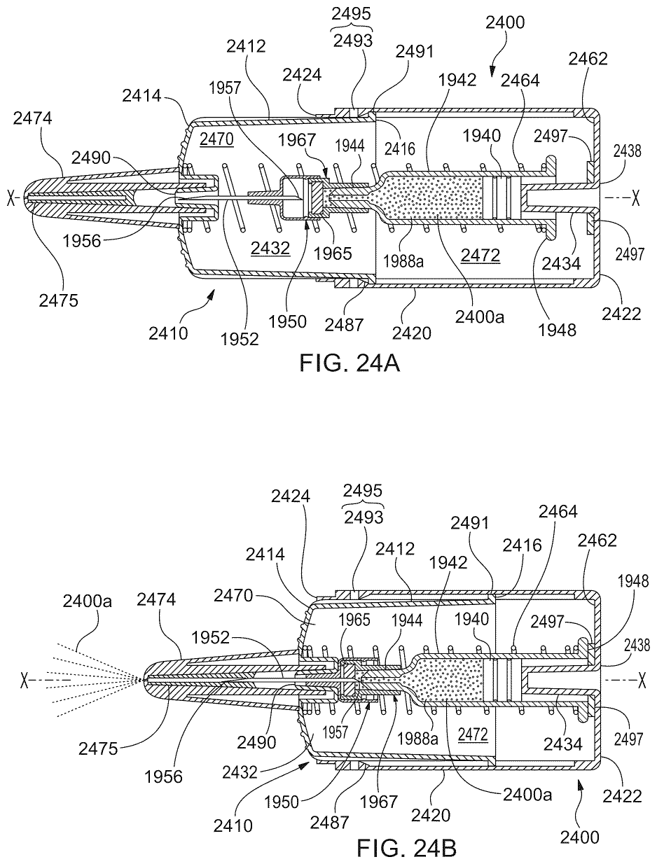

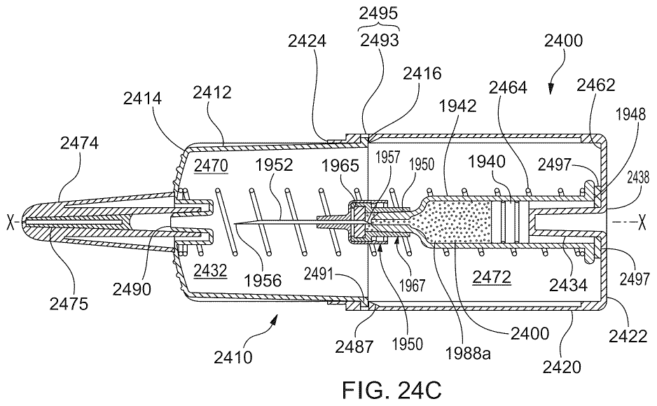

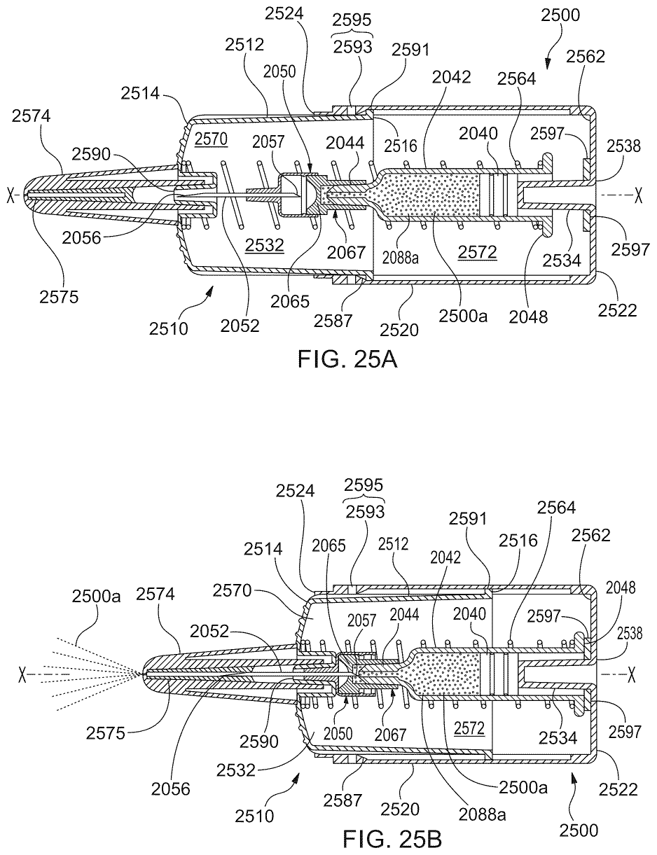

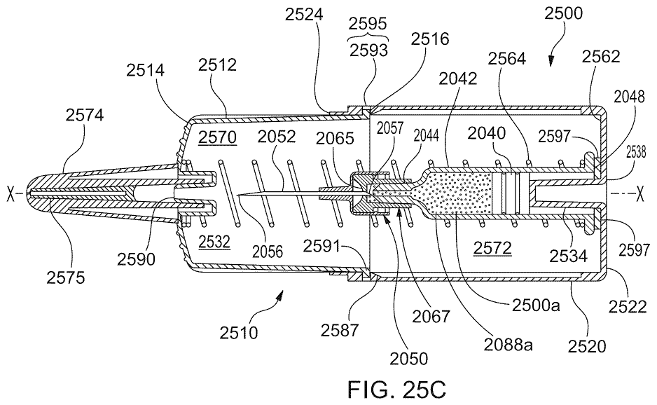

Referring now to the figures, wherein like elements are numbered alike throughout, FIGS. 1A-15, 20A-25C illustrate exemplary embodiments of a multimode nasal injector of the present disclosure capable of being converted to intramuscular or intravenous modalities.

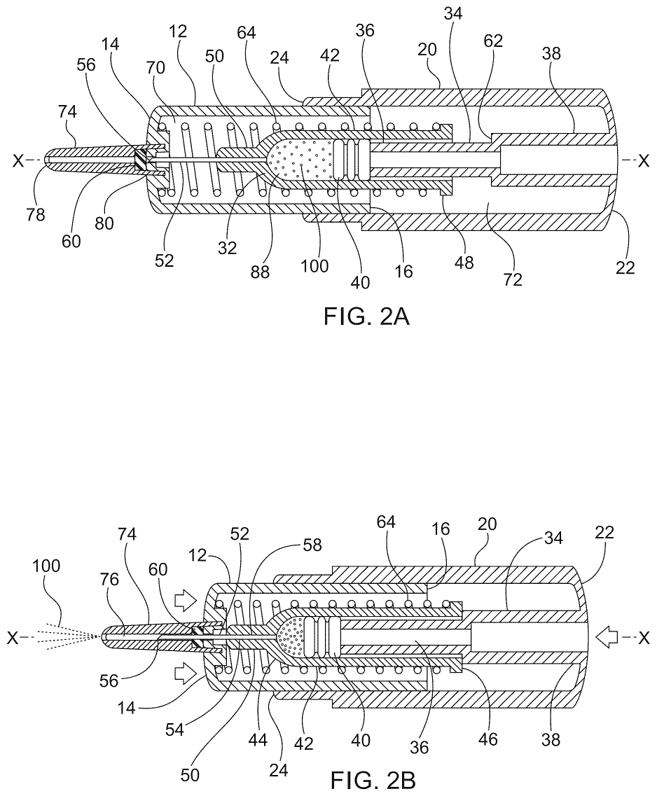

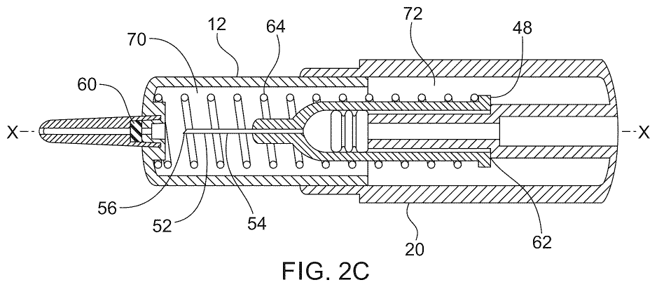

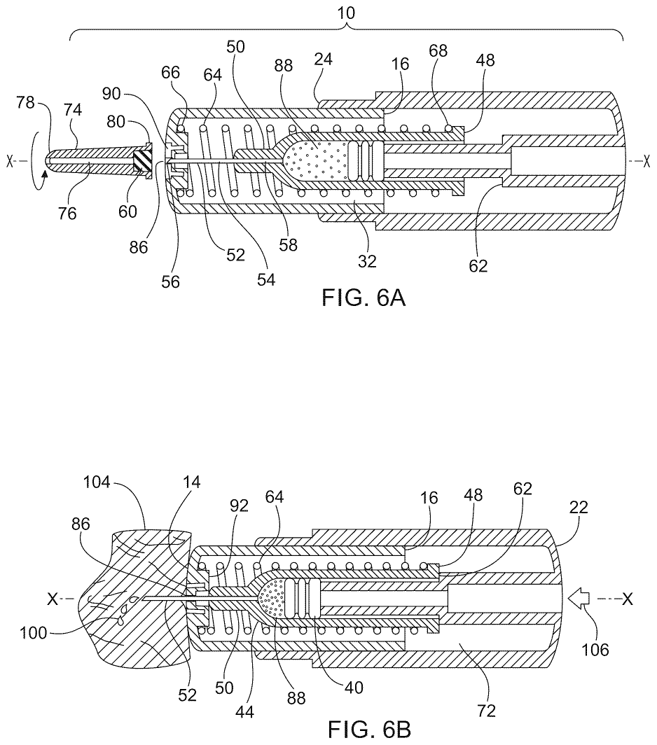

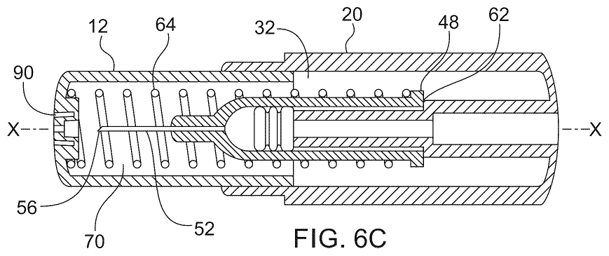

In one exemplary embodiment, the multimode nasal injector 10 includes a front housing 12 which has a front housing wall 18, with an outer surface and an inner surface, which circumscribes the front housing 12 between a front housing proximal end 14 and a front housing distal end 14, FIG. 2A. The front housing distal end 14 is closed and is fixed and removably coupled to a nasal nozzle 74 by a nozzle linkage 80 to an injector nozzle lock 90, FIG. 6A. The injector nozzle lock 90 may employ a number of structures to help removably couple the linkage 80 to the injector nozzle lock 90, such as: threads, tabs, snap fit latches, hook and latch, bayonet couplings, luer shaped heads, e.g., luer lock or luer slip, Morse taper, and hook and loop, for example. It is contemplated that in an exemplary embodiment the injector nozzle lock 90 will be a luer lock. A luer lock features a tapered male connector with external threads that twist into a female connector containing internal threads or lugs, forming a secure, leak-proof seal. This design combines frictional compression from the conical taper with mechanical locking from the threads. A luer lock is a secure syringe connection system that prevents leaks and accidental disconnections. With the luer lock, the nasal nozzle 74 is removed from the front housing 12 by twisting and removing it from the luer lock connector 90.

The front housing proximal end 14 is sized and shaped to fit and be slidably coupled to a rear housing 20, FIGS. 2A, 2B. The nozzle linkage 80 is disposed at one end of the nasal nozzle 74 and the nasal nozzle tip 78 at an opposing end. A canal 76 runs longitudinally from the linkage 80 to the nozzle tip 78 with openings at each end of the nasal nozzle 74 to allow movement of a medication 100 through the nasal nozzle 74, FIGS. 2A-2C.

The multimode nasal injector 10 also includes a rear housing 20 which has a rear housing wall 26, with an outer surface and an inner surface, between a distal end 24 and a proximal end 22 thereof, FIG. 1A. The rear housing proximal end 22 is closed, while the rear housing distal end 24 is open, which end 24 is sized and shaped to slidably couple with the front housing proximal end 14, FIG. 2A. In one exemplary configuration, the front housing proximal end 14 slidably couples inside the rear housing proximal end 22. In another exemplary configuration, the front housing proximal end 14 slidably couples outside the rear housing proximal end 22.

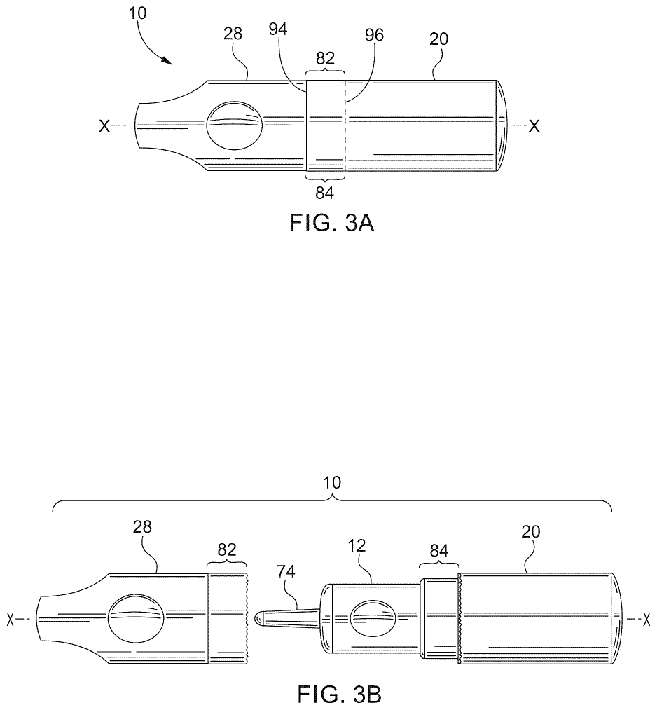

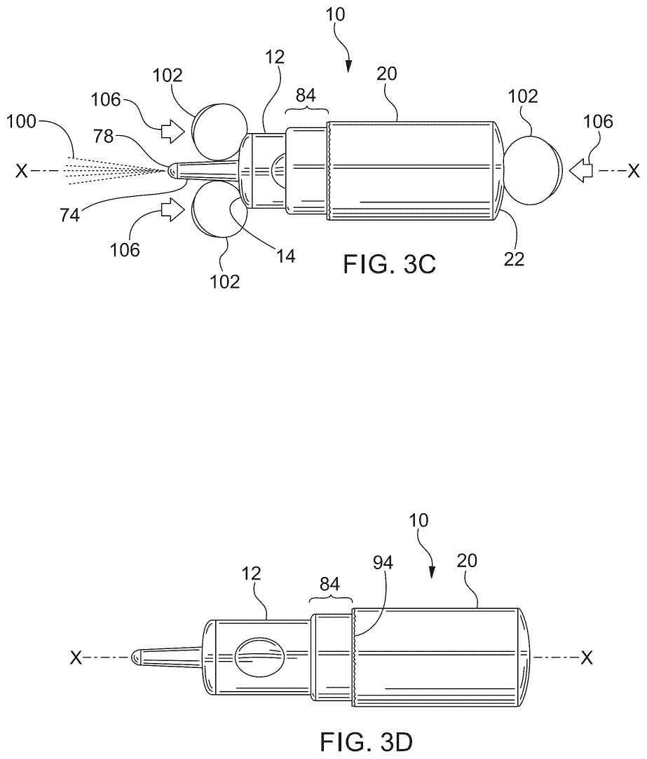

A cap 28 may be provided that is generally hollow with a closed end and an open end, FIGS. 1A, 1B. The open end of the cap 28 has a cap coupler 82 that is sized and shaped to fit over a portion of the rear housing distal end 24 referred to as the injector coupler 84, FIG. 1B. As shown in FIGS. 1A-2A, the multimode nasal injector 10 is in its initial state ready for use by a user. A label 94 (which could have printing such as logos, instructions, and warnings) may be wrapped around the rear housing 20 and the front cap 28, ensuring sterility as well as being tamper resistant, FIG. 3A. To remove the front cap 28, a multiplicity of perforations 96 may be positioned around the label 94 generally perpendicular to the injector's longitudinal axis X-X near an abutment of the front cap 28 and rear housing 20. The front cap 28 and rear housing 20 may be twisted relative to one another to break the seal and remove the front cap 28. It is contemplated that there could be other ways of attaching the cap 28 to the multimode nasal injector 10, such as a friction seal between the cap coupler 82 and the injector coupler 84, for example.

FIG. 1B schematically illustrates a top view of an exemplary embodiment of the multimode nasal injector 10. In this view the label 94 has been torn allowing cap 28 to be removed from the multimode nasal injector 10. This figure also shows the cap coupler 82 and the injector coupler 84 which are removably couplable from each other. The cap 28 may be rotated or pulled to remove it from the front housing 12 and away from the rear housing 20. The nasal nozzle 74 extends from the front housing distal end 14, and is enclosed within the cap 28 when the cap 28 is coupled with the multimode nasal injector 10.

FIGS. 2A-2C schematically illustrate the multimode nasal injector 10 as used in an intranasal (IN) application for applying medication 100 into a person's nasal passages. In this view, the relative placement of the rear housing 20 and front housing 12 (collectively the multimode nasal injector housing) in one embodiment are illustrated. The front housing 12 is removably coupled to a nasal nozzle 74 at the front housing distal end 14. This is accomplished by coupling the nasal nozzle 74 to an injector nozzle lock 90 at the front housing distal end 14, FIG. 6A. The front housing 12 has a wall 18 surface and an opposing front housing interior 70, where the front housing interior 70 is an open space, FIGS. 1A, 2A. Likewise, the rear housing 20 has a wall 26 surface and an opposing front housing interior 72, where the rear housing interior 72 is an open space.

A needle-based injection system in the form of a medication syringe assembly 32 is contained within the rear housing interior 72 and the front housing interior 70, FIG. 2A. The medication syringe assembly 32 is disposed in the front housing interior 70 and the rear housing interior 72. The medication syringe assembly 32 includes an elongated barrel 42 having a plunger 34 and a plunger seal 40 and medication 100 disposed therein. A needle 52 with a hub 50 is attached to the distal end of the syringe assembly 32. The syringe assembly 32 is a cylindrical tube having a barrel needle end 44 and a barrel plunger end 46, FIG. 2B. A selected amount of medication 100 is stored ready for use inside the barrel interior 88, between the barrel needle end 44 and the plunger seal 40. At or near an end of the syringe assembly 32 and opposite a hub 50 is a flange 48, or about the barrel plunger end 46, that extends from the outside of the syringe assembly 32 creating a shoulder. The syringe assembly 32 may be made from one or more of: glass, polypropylene, polyethylene, polycarbonate, acrylic (polymethyl methacrylate), nylon (polyamide), fluoropolymers (e.g. polytetrafluoroethylene), cyclic olefin copolymers, or cyclic olefin polymers or other suitable materials, for example.

A hub 50 circumscribes an aperture in the barrel needle end 44 to which the needle 52 is coupled. The needle 52 comprises a base 58, a lumen 54, and a tip 56, FIGS. 2A, 2B. Base 58 connects with hub 50 and needle 52 extends outwardly from base 58 away from syringe assembly 32. The lumen 54 is a hollow inside portion of the needle 52 that extends through the needle 52 from base 58 to tip 56, and through which the medication 100 is pushed by movement of the plunger 34 inside the syringe assembly 32 toward the barrel needle end 44.

The plunger seal 40 is disposed in contact with a plunger 34 at a distal end 36 of the plunger. A proximal, opposing proximal end 38 of the plunger is provided as an integral part of the rear housing 20 so the plunger and rear housing 20 are seamlessly connected as a unitary whole so the plunger 34 and rear housing 20 move together during use of the multimode nasal injector 10. Alternatively, the plunger 34 may be provided as a separately formed part which is subsequently permanently attached to the rear housing 20. Movement of the front and rear housings 12, 20 towards one another will cause the plunger 34 to move the plunger seal 40 towards the distal end of the syringe assembly 32, causing egress of medication 100 from the syringe assembly 32 through the needle 52.

The plunger seal 40 fits tightly (in order to create a liquid-tight seal) inside the syringe assembly 32. The plunger seal 40 is slidable, and creates a fluid-tight seal against, the inner surface of the syringe assembly 32 and tends to seal the medication 100 inside the barrel interior 88 and between the plunger seal 40 and the barrel needle end 44. The plunger seal 40 is generally made from rubber (natural or synthetic), latex, latex-free synthetic rubber, silicone rubber, thermoplastic elastomers, polyisoprene, butyl rubber, or similar materials chosen for being sterilizable, biocompatible, and with low friction for smooth plunger seal 40 movement inside the syringe assembly 32.

FIG. 2A illustrates the multimode nasal injector 10 in a first position with the front housing 12 extending away from the rear housing 20 in a first position of the multimode nasal injector 10. The multimode nasal injector 10 has a longitudinal axis X-X from the rear housing proximal end 22, to the nasal nozzle tip 78. User-supplied opposing forces along the X-X axis biases the rear housing proximal end 22 towards the front housing distal end 14 causing the front housing 12 to slidably move from a first position inwardly to a second position, FIG. 2B.

A spring 64 is contemplated to be a coiled compression type but other types of springs could be used such as compression springs, constant force springs, Belleville (disc) springs, volute springs, flat springs, gas springs, air springs, or elastomers and so forth, is disposed between the front housing distal end 14 and the flange 48. The spring 64 urges the front housing 12 outwardly and tends to keep the multimode nasal injector 10 in the first position. Force applied by a user to move the multimode nasal injector 10 to the second position must overcome the spring resistance, FIG. 2B, and when such force is released the spring 64 moves the multimode nasal injector 10 back to the first or lockout position, FIG. 2C.

FIG. 2B illustrates the multimode nasal injector 10 in the second position in which the front housing 12 is pushed back within the rear housing 20. Due to this movement, the needle 52 that is coupled to the medication syringe assembly 32 moves forward (relative to the front housing 12) and penetrates a self-sealing membrane 60, FIG. 6A, in the nozzle linkage 80 of the removable nasal nozzle 74. The movement also causes spring 64 to compress. The front housing 12 continues to move rearward and the rear housing 20 continues to move forward by squeezing front housing 12 and rear housing 20 together between the thumb and fingers. Upon sufficient compression, the inside surface of the front housing distal end 14, or injector lock base 92, FIG. 6B, engages with the front of the medication syringe assembly 32, or hub 50.

When the multimode nasal injector 10 is moved from the first position, FIG. 2A, to the second position, FIG. 2B, the spring 64 exerts force upon the flange 48. As a result, the plunger 34 and the plunger seal 40 move relative to the syringe assembly 32 inside of the barrel interior 88. The medication 100 is prefilled in barrel interior 88. The movement of the plunger seal 40 within the barrel urges the medication 100 out of the barrel interior 88 and through the hub 50. The medication 100 is further pushed through the needle lumen 54 to exit the needle tip 56.

When the front housing 12 is moved to the second position, the front housing distal end 14 is moved toward the needle tip 56, and the needle tip 56 extends through an aperture 86, FIG. 6A, in the front housing distal end 14 until, at the second position, the needle tip 56 is outside of the front housing distal end 14 by a predetermined length inside the nasal nozzle canal 76. The nasal nozzle canal 76 may be covered by, or partially filled with, a self-sealing membrane 60. The self-sealing membrane 60 allows the needle 52 to penetrate the self-sealing membrane 60, but before and after penetration by the needle 52, the self-sealing membrane 60 seals the interior of the front housing 12 limiting the exchange of contents of the front housing interior 70 and rear housing interior 72 from the environment outside the multimode nasal injector 10. The front housing 12 continues to push the barrel 42 back against the relatively unmoving plunger 34 inside the rear housing interior 72 which urges the medication 100 forward expelling a prescribed amount of medication 100 through and out of the needle 52.

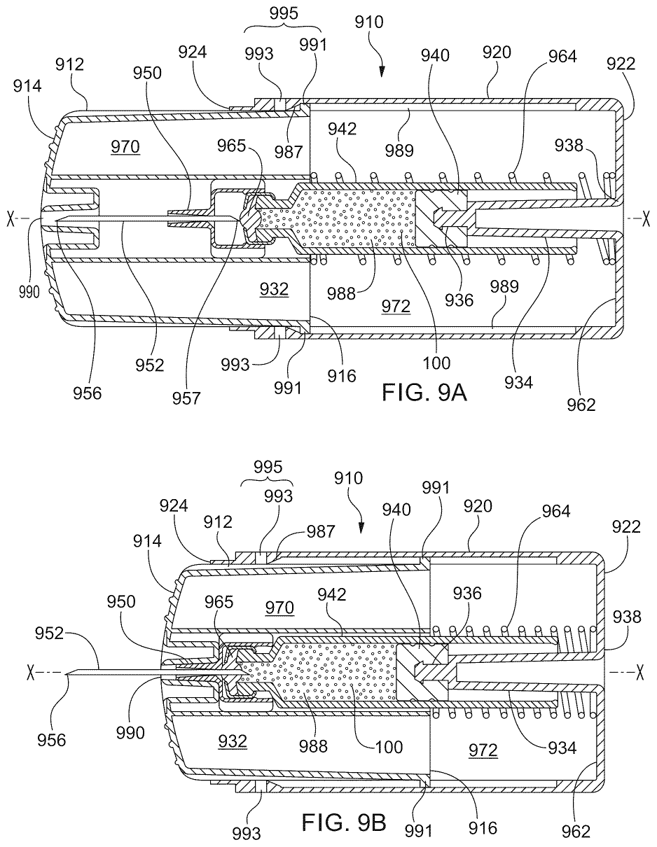

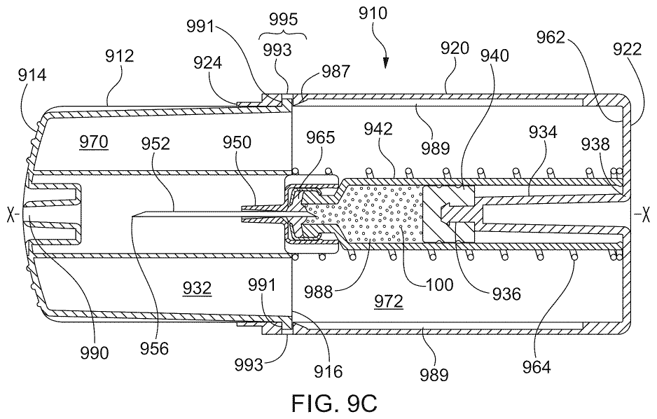

The multimode nasal injector 10 reaches the second position when the front housing 12 moves inside of the rear housing 20 until the flange 48 or the end of barrel plunger end 46 contacts the plunger stop 62, or the front end of the medication syringe assembly 32 contacts the inside of the front housing distal end 14. Alternatively, the plunger stop 62 may be the rear housing proximal end 22, FIG. 9C. The medication syringe assembly 32 is prefilled with medication 100 so during the movement of the multimode nasal injector 10 from the first position, FIG. 2A, to the second position, FIG. 2B, and with continued compression of the front housing 12 and the rear housing 20 the medicine 100 is transferred into the nasal nozzle 74, through the nasal nozzle canal 76 and then expelled out the nasal nozzle tip 78. The medication syringe assembly 32 moves rearward to contact a predetermined molded stop 62 on the plunger 34 and delivering the predetermined amount of medicament 100.

FIG. 2C illustrates that when the pressure is then released from the front housing 12 and rear housing 20, the spring 64 will expand returning to front the housing 12 and the rear housing 20 to a third position and locks out to prevent relative movement between the front and rear housings 12, 20. In the third position the needle 52 is held inside the front housing interior 70.

FIG. 3A illustrates an exemplary embodiment of the multimode nasal injector 10 as it is contemplated to be stored prior to use. The cap 28 is coupled to the rear housing 20 by the cap coupler 82 and to the injector coupler 84. FIG. 3A shows that the label 94 may be wrapped around the multimode nasal injector 10 in order to help hold the cap 28 in place, and pierced by perforations 96 in order to aid in tearing the label 94. The multimode nasal injector 10 has a longitudinal axis X-X. FIG. 3B illustrates the method of use of the injector is to remove the cap 28 and select the desired delivery piece, with or without nasal nozzle 74, appropriate for the desired method of delivery selected from intranasal, intramuscular, or intravenously.

FIG. 3C illustrates, for intranasal, the user should hold the multimode nasal injector 10 between fingers and thumb 102. One option for finger placement on the injector 10 is shown by the fingers 102. It is desirable that the user may use one hand and place two fingers 102 in position against the front housing distal end 14 and a thumb 102 against the rear housing proximal end 22. However, it should be recognized that this is not the only placement option. The user could use multiple hands, only fingers and no thumb, the fingers and/or thumb at varying positions, or some other body part or object on the multimode nasal injector 10. The objective is that the user can move the multimode nasal injector 10 from position one, FIG. 3B, to position two, FIG. 3C, to deliver the medication. In this manner, the medication 100 is delivered to the patient.

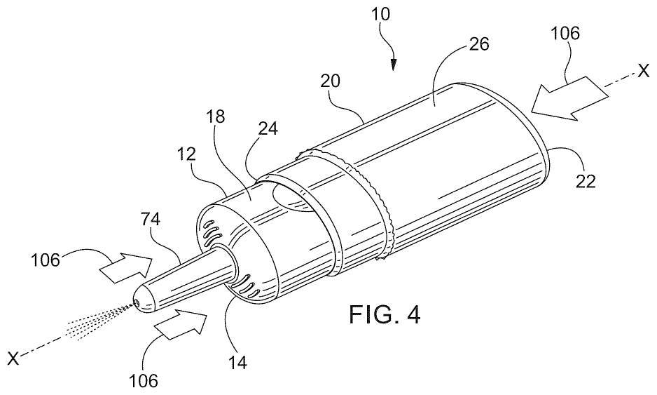

In order to move the multimode nasal injector 10 from position one to position two and deliver the medication 100, force must be applied along the longitudinal axis X-X as shown by the force directional arrows 106, FIG. 3C. FIG. 4 schematically shows a perspective view illustrating this point and more clearly shows the nasal nozzle 74 coupled with the front housing distal end 14. The multimode nasal injector 10 is being compressed to the second position with the front housing 12 being pressed upon by forces 106 along the X-X axis. The front housing wall 18 ingresses into the rear housing 20 through the rear housing distal end 24.

In intranasal use, the multimode nasal injector 10 will be in a first position with the front housing 12 proximal end inside or adjacent to the rear housing distal end 24. A first length of the front housing 12 extends from the rear housing 20. The user then inserts the nasal nozzle tip 78 and a portion of the nasal nozzle 74 into the patient's (which may be the user) nostril and squeezes the multimode nasal injector 10 along the longitudinal axis X-X, moving the multimode nasal injector 10 from the first position to a second position. Moving to the second position, the front housing proximal end 14 is moved further inside the rear housing 20 such that a second length of the front housing 12 extends from the rear housing distal end 24, where the first length is longer than the second length. Moving the multimode nasal injector 10 from the first position to the second position causes the active medication 100 to be pushed out of the multimode nasal injector 10, and the medication 100 will exit the barrel interior 88 and go through the needle lumen 54, nasal nozzle canal 76 and out the nasal nozzle tip 78 into the patient's nostril.

FIG. 3D illustrates, for intranasal, the return of the multimode nasal injector 100 to the first position. The spring 64 exerts force along the X-X axis in a direction opposite the force arrows 106. Thus when the users ceases to apply force in the direction of the force arrows 106, the front housing 12 is pushed back out of the rear housing 20 to generally the same length of the front and rear housings 12, 20 in the first position. However, the returned first position need not be the same internally. The label 94 (torn to allow for cap 28 removal) should not affect movement of the front housing 12. The cap 28 may be recoupled with the multimode nasal injector 10 by sliding it onto the injector coupler 84.

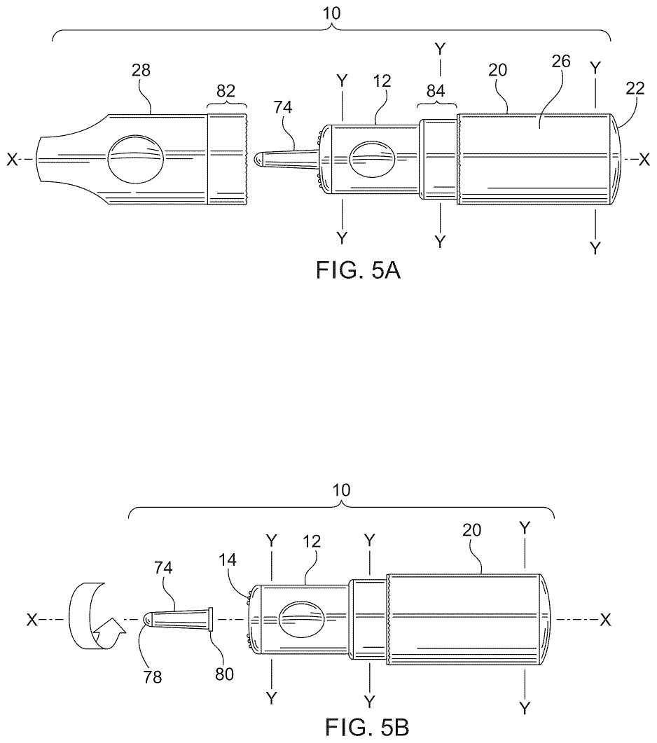

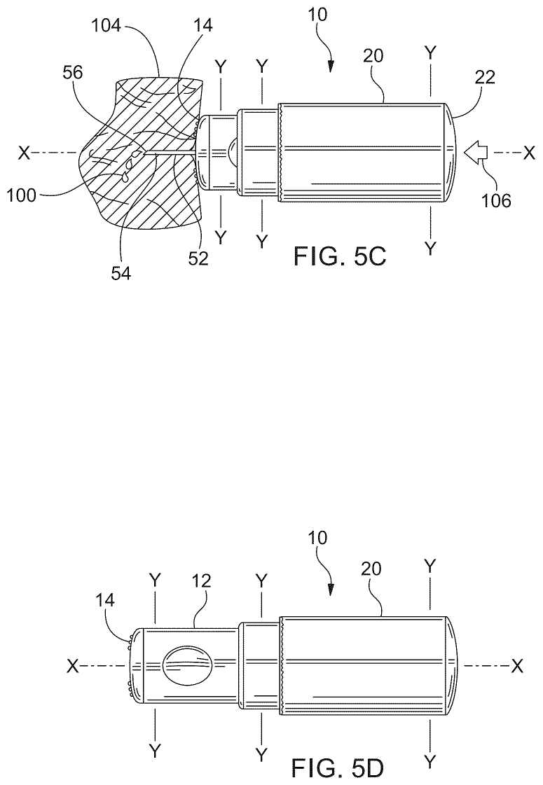

FIGS. 5A-5D illustrate the multimode nasal injector 10 when it is used for an intramuscular (IM) application for injecting medicament 100 via a needle 52 into a patient's thigh or tissue 104. FIG. 5A illustrates an initial action for using the multimode nasal injector 10 for intravenous injections by breaking the label 94 if necessary and removing the cap 28 by disengaging the cap coupler 82 from the injector coupler 84. The cap 28 can be completely removed from the multimode nasal injector 10 and discarded or kept for later. The multimode nasal injector 10 is in the first position, and relative positions of the multimode nasal injector 10 are noted by longitudinal markers Y. FIG. 5B shows that the nasal nozzle 74 is removed from the front housing distal end 14. In the illustrated embodiment, the nasal nozzle 74 is twisted to disengage it from the front housing distal end 14.

FIG. 5C shows the front housing distal end 14 of the multimode nasal injector 10 pushed against the thigh (or other tissue) 104 and is thus generally stationary while the rear housing 20, continues to be pressed forward toward the tissue 104 with the direction of force 106 along the X-X axis. The needle tip 56 and a length of the needle 52 are exposed outside of the front housing interior 70, exiting from the front housing proximal end 14. As the needle 52 is exposed, it penetrates into the thigh 104 or other tissue. In an exemplary embodiment, the needle 52 is extended and penetrates the tissue 104 generally 0.625 inches. At this point of the travel, the interior side of the front housing 12 engages and abuts against the front end of the barrel 42 of the medication syringe assembly 32. In FIG. 5C, the multimode nasal injector 10 is in the second position and relative positions of the multimode nasal injector 10 are noted by longitudinal markers Y, which can be compared to their positions in the first position of FIG. 5A.

FIG. 5D further illustrates the multimode nasal injector 10 removed from the thigh after the medication is administered. The front housing 12 returns to its original position via a biasing force exerted by a spring 64 and holds the needle 52 inside the front housing interior 70, a “safe mode” or “lock-out mode”, minimizing the chance of an unintended, secondary needle puncture. In FIG. 5D, the multimode nasal injector 10 is in the return, first position, and relative positions of the multimode nasal injector 10 are noted by longitudinal markers Y, which can be compared to their positions in the first position, FIG. 5A and second position, FIG. 5C.

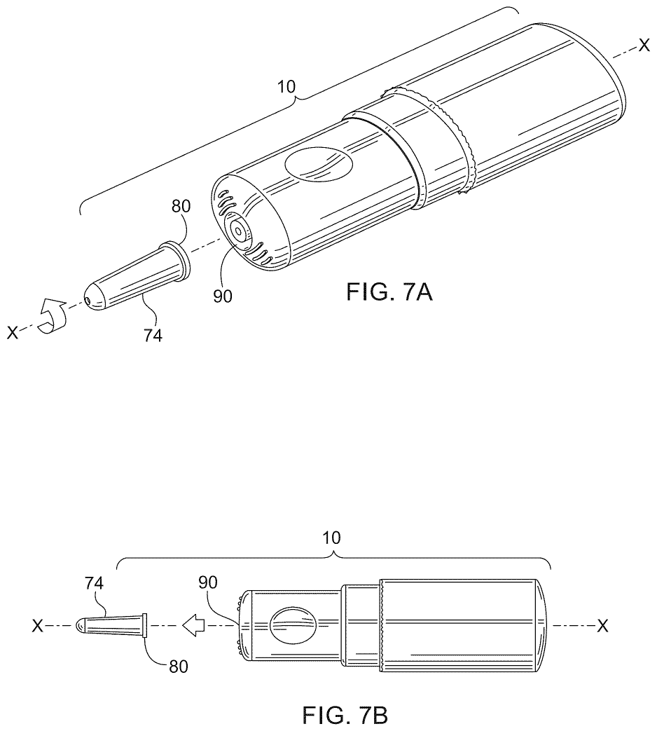

FIG. 6A-6C are similar to FIGS. 5A-5D but show a longitudinal sectional view of the intramuscular (IM) application of the multimode nasal injector 10. FIG. 6A illustrates that the nasal nozzle 74 is removed from the front housing distal end 14 after the cap 28 has been removed. Prior to use, the multimode nasal injector 10 is in the first position, FIG. 6A. The nasal nozzle 74 is removed from the front housing 12 and the nasal nozzle 74 may be discarded, FIGS. 6A, 7A, 7B. FIG. 6B shows that when the multimode nasal injector 10 is pushed, the front housing 12 is moved from the first position to the second position. When the multimode nasal injector 10 is pushed against the patient's tissue, the front housing 12 starts against the tissue in the first position. Force applied along the longitudinal axis X-X of the injector moves a portion of the front housing into the rear housing 20 and to the second position. In the same movement, the needle 52 is exposed from the interior of the front housing 12 out of the distal end 14 and inserted into the tissue 104 and the spring 64 is compressed, storing elastic potential energy in the spring 64. Thus, the movement toward the tissue 104 by the multimode nasal injector 10 results in the tissue blocking movement forward by the front housing 12 toward the tissue 104 but the pressure causes the needle 52 to be exposed and inserted into the tissue 104.

More specifically, the medication syringe assembly 32 is coupled to the rear housing 20. The front housing's movement from the first position toward the second position moves it closer to the medication syringe assembly 32 which causes needle tip 56 and needle 52 to be exposed through aperture 86 out of the front housing distal end 14, and be pushed into the tissue 104 of the patient. Once the hub 50 engages the injector lock base 92, the syringe assembly 32 begins moving toward the rear housing proximal end 22. The movement of the syringe assembly 32 toward the plunger stop 62 causes the barrel interior 88 to be decreased in volume. Medication 100 held in the barrel interior 88 is forced from the barrel interior 88, through the hub 50, through the needle lumen 54, and into the tissue 104 of the patient. The second position is reached, and internal movement generally ceases when the flange 48 engages the plunger stop 62.

The length of the needle 52 may be chosen such that the needle 52 will extend from the front housing distal end 14 by a chosen length so as to best deliver the medication 100 into the tissue 104 at a desired depth. In one embodiment, the needle 52 extends from the front housing distal end 14 a given length of generally 0.625 inches from front housing distal end 14 to needle tip 56.

FIG. 6C illustrates the multimode nasal injector 10 and needle 52 removed from the tissue 104. The spring 64 is positioned between the interior side of the front housing distal end 14 and the flange 48 opposite the plunger stop 62. Then when the pressure exerted by the user is relaxed, the spring's stored elastic potential energy is released which moves the front housing 12 back to the first position. However, the spring 64 continues to urge the flange 48 against the plunger stop 62, so the medication cartridge may not move to its original position. Thus, the front housing 12 moves out and the needle 52 ends up inside the front housing interior 70 and into a locked out position protecting users from receiving an unintended puncture. In another use, the needle 52 may be inserted into an injection port (not shown) of an intravenous (IV) set instead of tissue 104 via a luer connection.

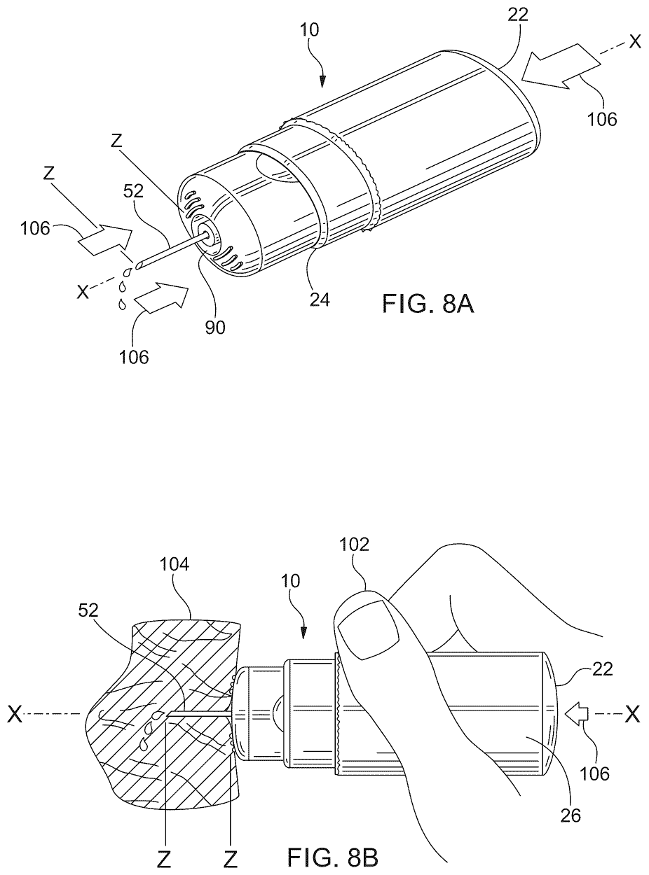

FIGS. 8A and 8B show the multimode nasal injector 10 in perspective view during use in the IM injection mode of FIGS. 7A-7C. FIG. 8A shows the directions of force 106 applied to the multimode nasal injector 10 along the X-X axis in order to move it to the second position, FIG. 8B. In the second position, the needle 52 is exposed outside the multimode nasal injector 10 by a length Z-Z from needle tip 56 to injector nozzle lock 90. FIG. 8B shows the multimode nasal injector 10 being compressed to the second position with the front housing 12 being pressed upon by forces 106 along the X-X axis. The front housing wall 18 ingresses into the rear housing 20 through the rear housing distal end 24. The multimode nasal injector 10 may be held by the rear housing wall 26 nearer the rear housing proximal end 22 for easier operation. The front housing distal end 14 is pushed in the direction of force 106 along the longitudinal axis X-X of the multimode nasal injector 10 against the tissue 104 of the patient where injection of the medication 100 is desired. Typically, the multimode nasal injector 10 is pushed against a thigh 104 of the patient. The user may grip the multimode nasal injector 10 with fingers 102 or anything sufficient to push the multimode nasal injector 10 against the tissue 104 and move the multimode nasal injector 10 to the second position.

In an additional exemplary embodiment, FIGS. 9A-15, a multimode nasal injector 910 is provided which is similar in many respects to the embodiment of FIGS. 1A-8B. Similar parts and structures with similar function are noted in Table 1 at the end of this section, where a “900”-series reference numeral is used for structures in FIGS. 9A-15. Notable differences between the multimode nasal injector 910 and the injector 10 are described in more detail below and include: a double-ended needle 952 disposed in spaced apart relation from the cartridge 932 prior to injection; medication spacers 997a, 997b for limiting the range of movement of the front and rear housings 912, 920 (collectively multimode nasal injector housing) the relative to one another so as to limit the amount of medication 100 expelled; a plunger stop located near the rear wall 922 of the rear housing 920; and a more detailed view of an exemplary lock out structure 995 that can be used throughout all embodiments 10, 910 disclosed herein. Further differences and similarities between injector 10 and injector 910 are noted below.

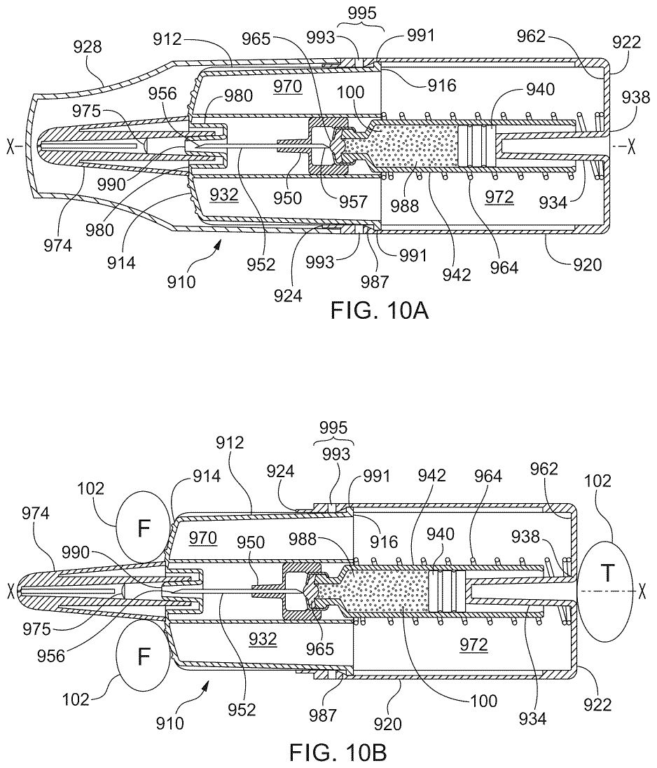

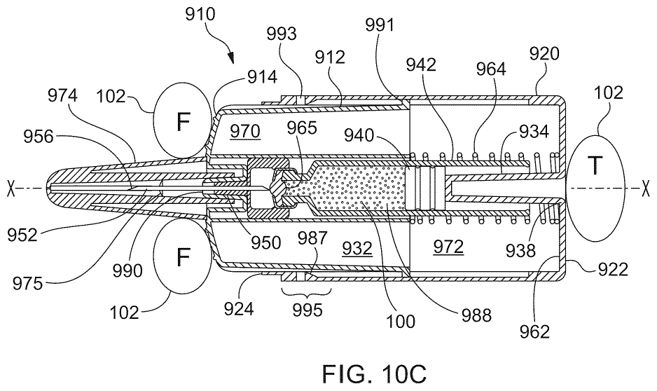

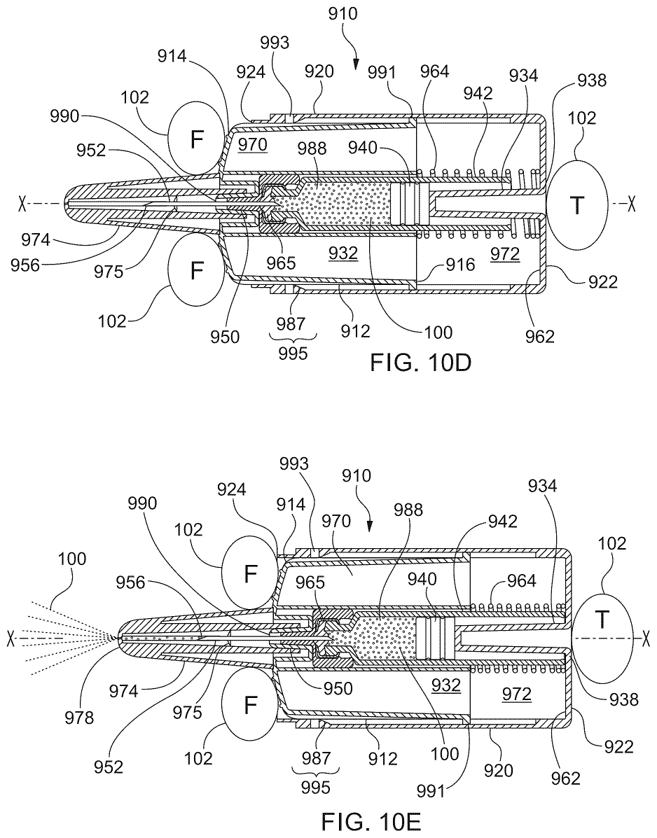

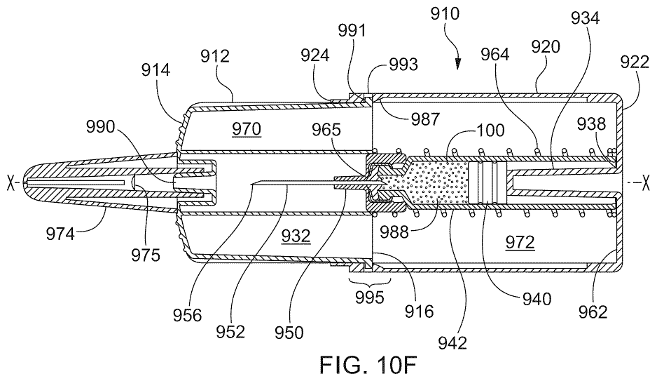

The multimode nasal injector 910 includes a front housing 912 with a front housing proximal end 916 and a front housing distal end 914, FIG. 9A. The rear housing proximal end 922 is closed with plunger 934 disposed thereat, while the rear housing distal end 924 is open, which end 924 is sized and shaped to slidably couple with the front housing proximal end 916, FIG. 9A. In one exemplary configuration, the front housing proximal end 916 slidably couples inside the rear housing distal end 924. In another exemplary configuration, the front housing proximal end 916 slidably couples outside the rear housing distal end 924. A cap 928 may be provided that is generally hollow with a closed end and an open end, FIG. 10A, 15. The cap 928 be provided in the same form, and function in the same manner, as the cap 28 previously described. It is understood that the multimode nasal injector 910 may be provided with a nasal nozzle 974, FIG. 10A, but is omitted from FIGS. 9A-9C to more clearly show the lockout structure 995.

The lockout structure 995 is provided to lock the injector 910 after it has been used so that the front housing 912 can no longer be moved relative to the rear housing 920 and so that the double-ended needle 952 is securely retained within the injector 910 to minimize puncture by the double-ended needle 952 after use. In this regard, the lockout structure 995 can include a lockout tab 991 disposed on the front housing 912, FIG. 9A. Alternatively the tab 991 may be provided on the rear housing 920. As the front and rear housings 912, 920 are urged together, the tab 991 travels along a channel 989 in the rear housing 920, FIG. 9B. When the front and rear housings 912, 920 are released, the stored elastic potential energy in the spring 964 pushes the front and rear housings 912, 920 apart from each other with sufficient force that the lockout tab 991 rides up over a shoulder 987 at a distal end of the channel 989 to rest in a lockout slot 993, FIG. 9C. (Alternatively, the slot 993 may be located on the front housing 912 when the tab 991 is located on the rear housing 920.) Once the lockout tab 991 is located in the lockout slot 993 the front and rear housings 912, 920 are immobile relative to one another with the double-ended needle 952 safely contained in the injector 910.

Turning to the double-ended needle feature, FIG. 9B illustrates the front housing 912 pushed back within the rear housing 920. Due to this movement, the double-ended needle 952 that is coupled to the medication cartridge 932 moves forward (relative to the front housing 912) and a proximal tip 957 of the double-ended needle 952 penetrates a self-sealing membrane 965, FIG. 9B, putting the double-ended needle 952 in fluid communication with the medication 100. The movement also causes the spring 964 to compress exerting force upon the rear housing distal end 924. The front housing 912 continues to move rearward and the rear housing 920 continues to move forward by squeezing front housing 912 and rear housing 920 together. Upon sufficient compression, medication 100 is expelled from the injector 910 due to movement of the plunger ¥27 seal 940 within the barrel urging the medication 100 out of the barrel interior 988 and through the hub 950. The medication 100 is further pushed through the needle lumen to exit the distal needle tip 956.

The spring 964 is contemplated to be a coiled compression type but other types of springs could be used such as compression springs, constant force springs, Belleville (disc) springs, volute springs, flat springs, gas springs, air springs, or elastomers and so forth, is disposed between the front housing distal end 914 and the rear housing distal end 924. The spring 964 urges the front housing 912 outwardly and tends to keep the multimode nasal injector 910 in the first position. Force applied by a user to move the multimode nasal injector 910 to the second position must overcome the spring resistance, FIG. 9B, and when such force is released the spring 964 moves the multimode nasal injector 910 back to the lockout position, FIG. 9C.

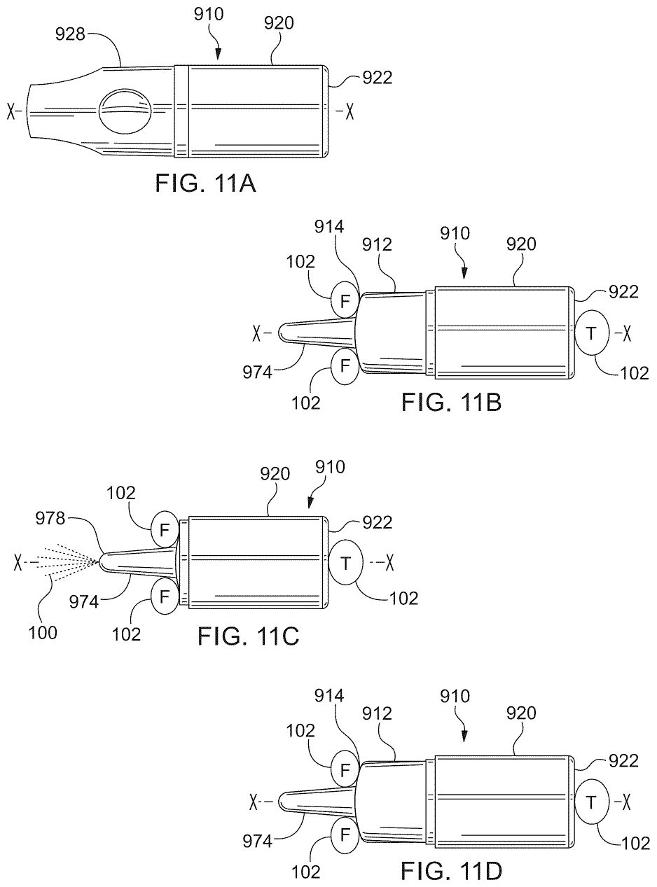

Use of the multimode nasal injector 910 intranasally is shown in cross-sectional views in FIGS. 10A-10F and in top view in FIGS. 11A-11D. The front housing distal end 914 is closed and is fixed and removably coupled to a nasal nozzle 974 by a nozzle linkage 980 to an injector nozzle lock 990, FIG. 10A. The injector nozzle lock 990 may employ a number of structures to help removably couple the linkage 980 to the injector nozzle lock 990, such as: threads, tabs, snap fit latches, hook and latch, bayonet couplings, luer shaped heads, and hook and loop. It is contemplated that in an exemplary embodiment the injector nozzle lock 990 will be a luer lock. The nozzle linkage 980 is disposed at one end of the nasal nozzle 974 and the nasal nozzle tip 978 at an opposing end. A canal runs longitudinally through the nasal nozzle 974 to allow movement of a medication 100 through the nasal nozzle 974, FIG. 10E.

The needle-based injection system, provide in the form of a medication cartridge 932, is contained within the rear housing interior 972 and the front housing interior 970 and is affixed in the front housing interior 970 of the rear housing 920, FIG. 10A. The medication cartridge 932 includes a plunger 934 and a plunger seal 940 and medication 100 disposed therein. A double-ended needle 952 with a hub 950 is attached to the distal end of the cartridge 932 and extends outwardly away from the cartridge 942. The double-ended needle 952 includes a proximal tip 957 and a distal tip 956. A selected amount of medication 100 is stored ready for use inside the barrel interior 988. A self-sealing membrane 965 is disposed at a distal end of the cartridge 942 proximate a proximal tip 957 of the double-ended needle 952. The plunger seal 940 fits tightly inside the cartridge 942. The plunger seal 940 is slidable and creates a fluid-tight seal against the inner surface of the cartridge 942 and tends to seal the medication 100 inside the barrel interior 988 and between the plunger seal 940 and the self-sealing membrane 965. The plunger seal 940 is generally made from rubber (natural or synthetic), latex, latex-free synthetic rubber, silicone rubber, thermoplastic elastomers, polyisoprene, butyl rubber, or similar materials chosen for being sterilizable, biocompatible, and with low friction for smooth plunger seal 940 movement inside the cartridge 942.

The plunger seal 940 is disposed in contact with a plunger 934 at a distal end 936 of the plunger 934. A proximal, opposing proximal plunger end 938 of the plunger 934 is provided as an integral part of the rear housing 920 so the plunger 934 and rear housing 920 are seamlessly connected as a unitary whole, so the plunger 934 and rear housing 920 move together during use. Alternatively, the plunger 934 may be provided as a separately formed part which is subsequently permanently attached to the rear housing 920. Movement of the front and rear housings 912, 920 towards one another will cause the plunger 934 to move the plunger seal 940 towards the distal end of the cartridge 942. At the same time movement of the front and rear housings 912, 920 towards one another will cause the double-ended needle 952 to pierce the self-sealing membrane 965 to place the double-ended needle 952 in fluid communication with the medication 100, FIG. 10D. Continued movement will cause medication 100 to pass from the cartridge 942 through the double-ended needle 952 and out from the injector 910, FIG. 10E. To deter backflow of medication into the injector housing 970, a canal seal 975 may be provided in the canal of the nasal nozzle 974, FIGS. 10A-10F. Once the canal seal 975 is pierced by the distal needle tip 956 of the needle 952, the canal seal 975 provides a fluid-tight seal around the needle 952, FIG. 10E. A canal seal 975 may be provided in any nasal nozzle, e.g. nozzles 74, 974, 2174-2574, of any embodiment of the present invention.

FIGS. 10A, 10B illustrate the multimode nasal injector 910 in a first position with the front housing 912 extending away from the rear housing 920 in a first position. The multimode nasal injector 910 has a longitudinal axis X-X from the rear housing proximal end 922 to the nasal nozzle tip 978. User-supplied opposing forces along the longitudinal axis X-X bias the rear housing proximal end 922 towards the front housing distal end 914 causing the front housing 912 to slidably move from a first position inwardly, FIGS. 10C-10D, to expel the medication 100, FIG. 10E.

In intranasal use, the multimode nasal injector 910 will be in a first position with the front housing 912 proximal end inside or adjacent to the rear housing distal end 924, FIGS. 10A, 11B. A first length of the front housing 912 extends from the rear housing 920. The user then inserts the nasal nozzle tip 978 and a portion of the nasal nozzle 974 into the patient's (which may be the user) nostril and squeezes the multimode nasal injector 910 along the longitudinal axis X-X, moving the multimode nasal injector 910 from the first position to a second position, FIGS. 10D, 11C. Moving to the second position, the front housing proximal end 916 is moved further inside the rear housing 920 such that a second length of the front housing 912 extends from the rear housing distal end 924, where the first length is longer than the second length. Moving the multimode nasal injector 910 from the first position to the second position causes the active medication 100 to be pushed out of the multimode nasal injector 910, and the medication 100 will exit the barrel interior 988 and go through the needle lumen, nasal nozzle canal and out the nasal nozzle tip 978 into the patient's nostril.

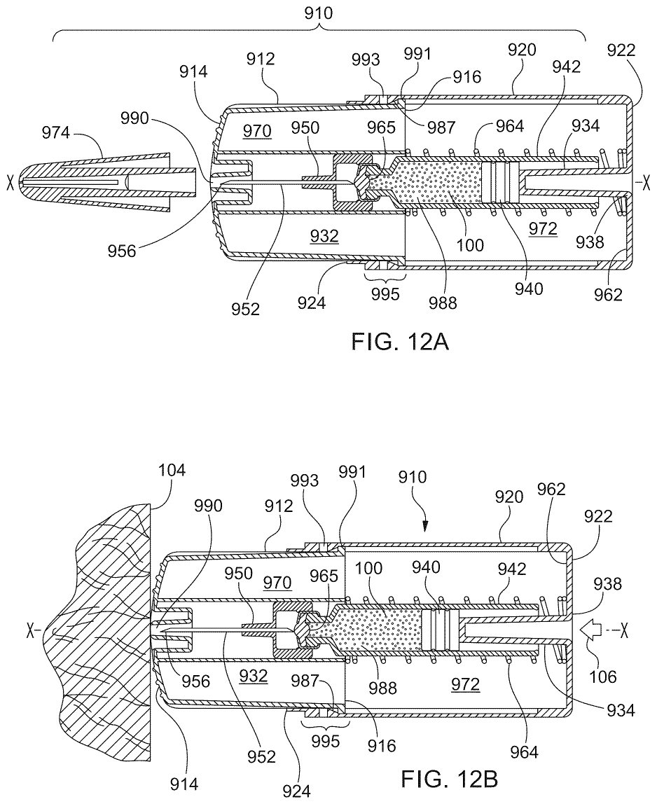

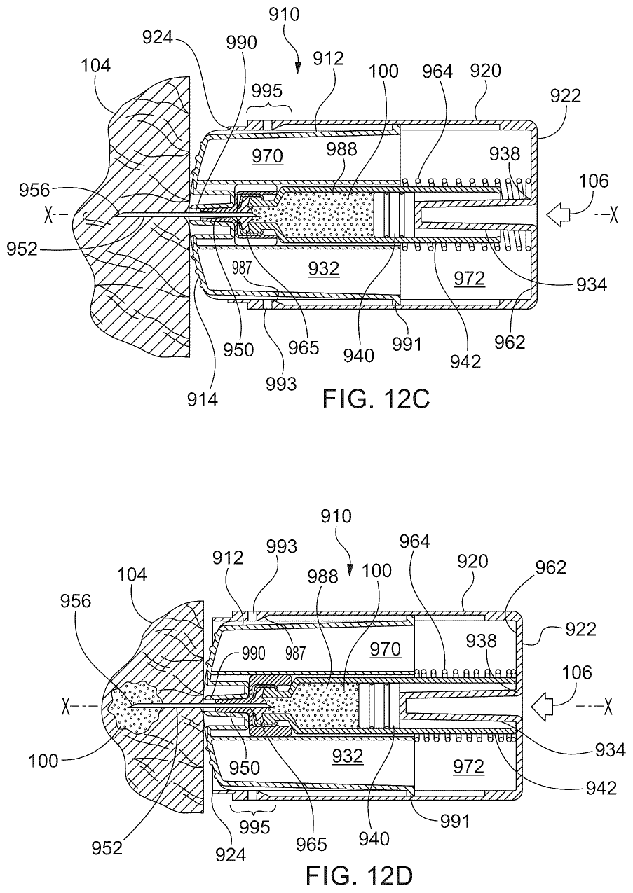

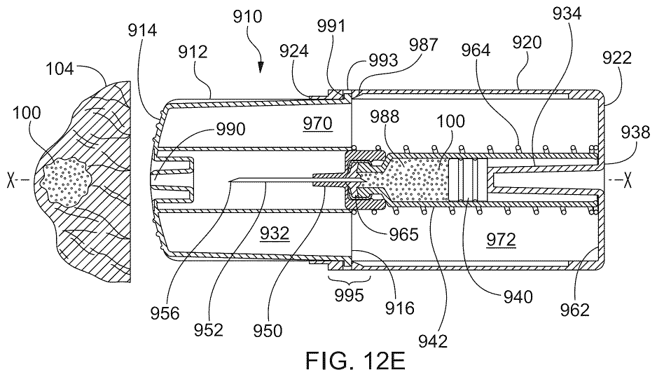

FIG. 12A-12E illustrate the multimode nasal injector 910 when it is used for an intramuscular (IM) application for injecting medicament 100 via a double-ended needle 952 into a patient's thigh or tissue 104. FIG. 12A illustrates an initial action for using the multimode nasal injector 910 for intramuscular injections: removing the nasal nozzle 974. FIG. 12B shows the front housing distal end 914 of the multimode nasal injector 910 pushed against the thigh (or other tissue) 104 and is thus generally stationary while the rear housing 920, continues to be pressed forward toward the tissue 104 with the direction of force 106. The distal needle tip 956 and a length of the double-ended needle 952 are exposed outside of the front housing interior 970, exiting from the front housing proximal end 916. As the double-ended needle 952 is exposed, it penetrates into the thigh 104 or other tissue. At the same time the needle 952 pierces the self-sealing membrane 965. In an exemplary embodiment, the double-ended needle 952 is extended and penetrates the tissue 0.625 inches. At this point of the travel, the interior side of the front housing 912 engages and abuts against the front of the pre-filled cartridge 942 of the medication cartridge 932. The interior side of the front housing 912 continues to push the barrel back against the relatively unmoving plunger 934 inside the rear housing interior 972 which urges the medication 100 forward expelling a prescribed amount of medication 100 through and out of the double-ended needle 952 and into the tissue 104, FIG. 12D.

FIG. 12E further illustrates the multimode nasal injector 910 removed from the thigh 104 after all the medication is administered. The front housing 912 returns to a lockout position via a biasing force exerted by the spring 964 and holds the double-ended needle 952 inside the front housing interior 970, a “safe mode” or “lock-out mode”, minimizing the chance of an unintended, secondary needle puncture.

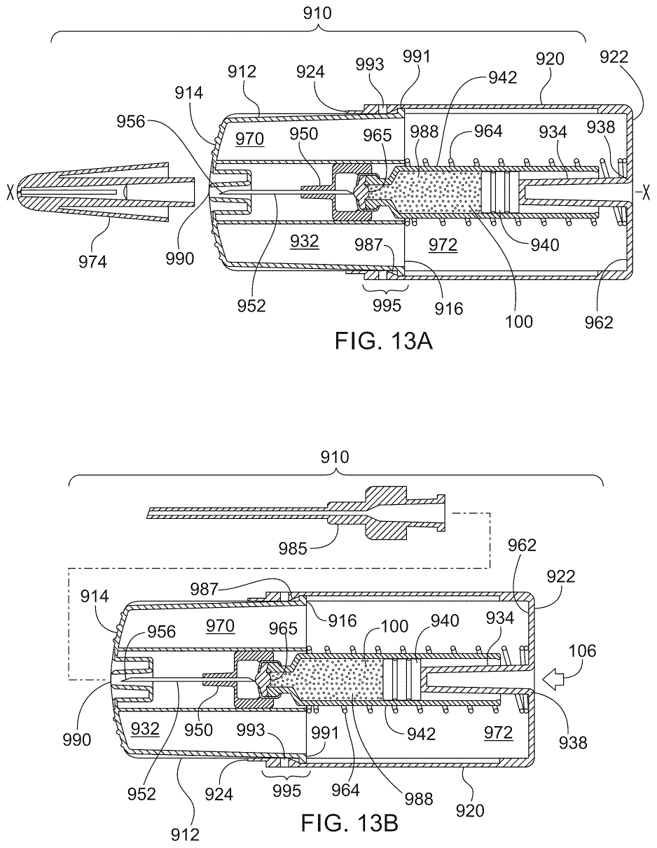

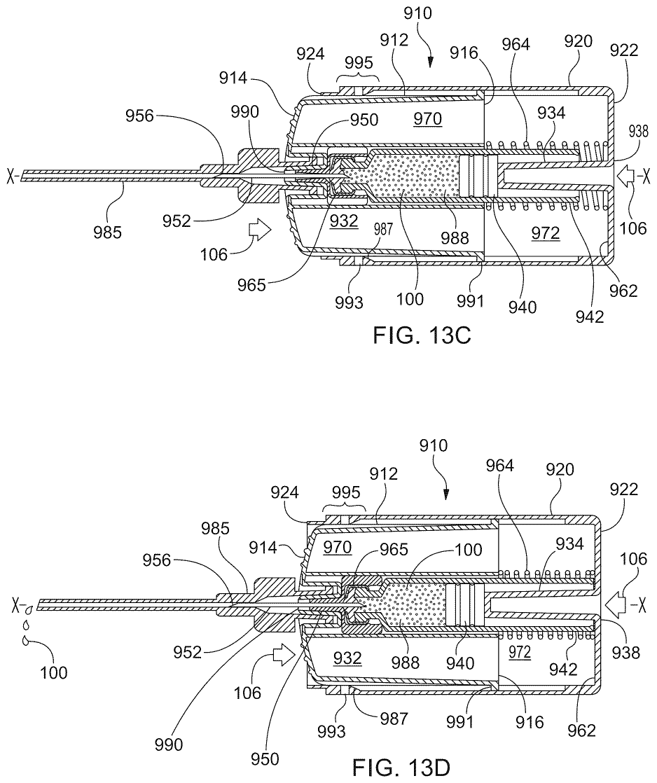

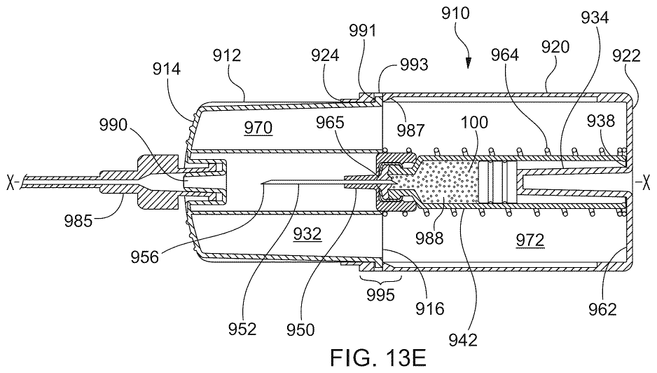

In another use, the double-ended needle 952 may be inserted into the injection port of an intravenous (IV) set (not shown) or an IV picc line 985, FIGS. 13A-13E. FIG. 13A illustrates the initial action for adapting the multimode nasal injector 910 for intravenous injections: removing the nasal nozzle 974. FIG. 13B shows an IV picc line 985 proximate the front housing distal end 914 of the multimode nasal injector 910. The picc line 985 is attached to the injector 910, FIG. 13C, at the hub 950 and the housings 912, 920 compressed together with the double-ended needle 952 in fluid communication with the medication 100. The distal needle tip 956 extends into the picc line 985 and medication travels through the line 985, FIG. 13D. FIG. 13E illustrates the multimode nasal injector 910 after the medication is administered. The front housing 912 returns to a lockout position via a biasing force exerted by a spring 964 and holds the double-ended needle 952 inside the front housing interior 970, a “safe mode” or “lock-out mode”, minimizing the chance of an unintended, secondary needle puncture.

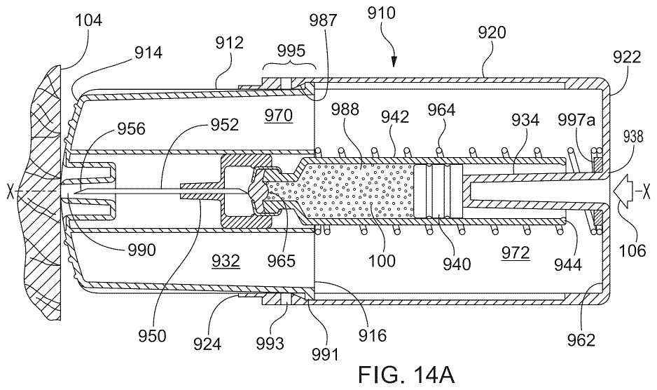

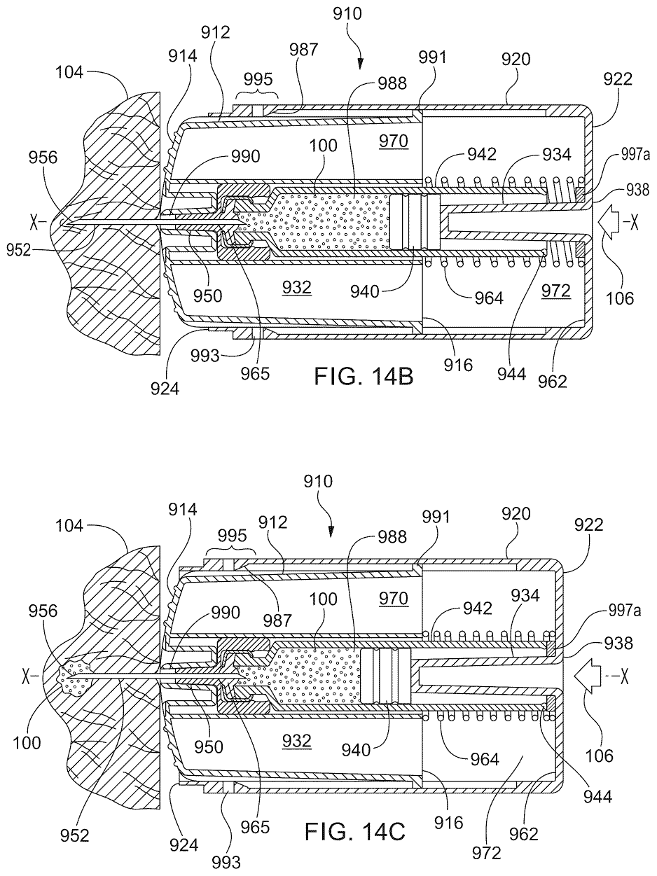

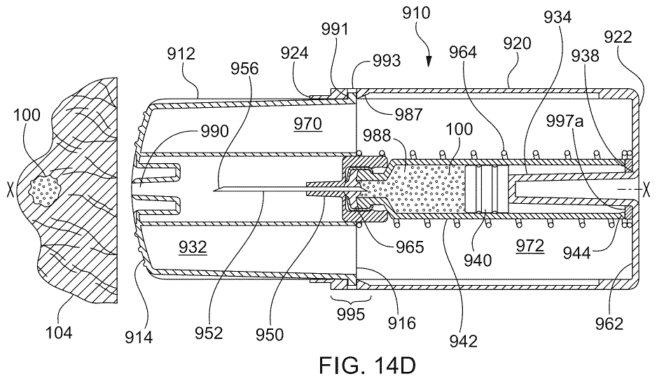

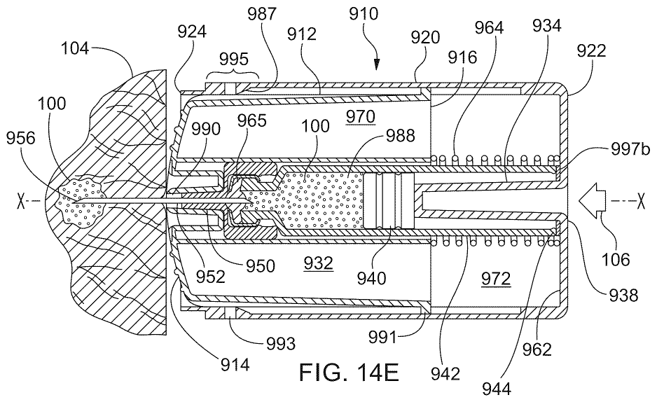

FIGS. 14A-14E show the multimode nasal injector 910 during use in the IM injection mode with medication spacers 997a-997b (collectively 997) in place to define the amount of medication to be delivered. FIGS. 14A-14D show the injection sequence from start, FIG. 14A, to completion at injector lockout, of FIG. 14D. A relatively thicker medication spacer 997a, having a thickness measured along the direction of the longitudinal axis X-X, is disposed internal to the injector 910 at the rear housing proximal end 922. The spacer 997a limits the range of motion of the front and rear housings 912, 920 towards one another, which limits the range of travel of the plunger seal 940, which in turn limits the amount of medication 100 dispensed. As the two housings 912, 920 approach one another movement is curtailed when the barrel plunger end 946 abuts the spacer 997a, FIG. 14C. Medication spacers of differing thicknesses can limit the range of travel differently, and thus the amount of medication dispensed. For example, a relatively thinner spacer 997b than that of spacer 997a will allow greater range of motion and increased medication delivery, FIG. 14E. The medication spacers 997a-997b can be applied over the plunger 934 before assembly in the rear housing 920. This way the cartridge filler may fill all cartridges 932 with only one volume of medication and need not be precise, (e.g. can vary by +1 ml). This approach with use of the spacers 997a-997b can be cheaper, faster and more precise. For example, without any spacer in place, the rear housing 920 can be designed and dimensioned to deliver a 0.4 ml injection due to the travel of the cartridge in relationship to the rear housing 920. If a lesser dosage volume, e.g. 0.3 ml is desired, a spacer 997b is assembled in place reducing the travel so only 0.3 ml is administered during injection. If an even lesser dosage volume, e.g. 0.13 ml is desired, a spacer 997a is assembled in place reducing the travel so only 0.13 ml is administered.

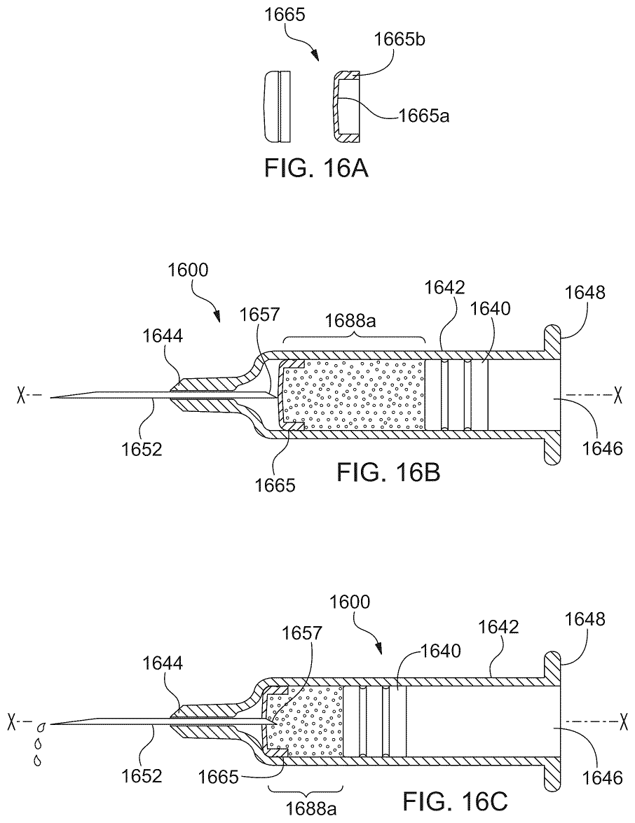

In a further of its aspects, the present disclosure provides various needle-based injection systems (NIS) in the form of syringes, where the syringes include the important feature that the needle is disposed out of contact with the medication prior to use, i.e., the needle is isolated from the medication, FIGS. 16A-20C. One exemplary embodiment of such a medication syringe 1600 is illustrated in FIGS. 16A-16C. The syringe 1600 has an elongated barrel 1642 having a barrel plunger end 1646 and an opposing barrel needle end 1644 and a longitudinal axis X-X extending therebetween. A flange 1648 is provided at the barrel plunger end 1646. The barrel 1642 has a cavity therein for holding a medication 1688. A plunger seal 1640 is slidably disposed in the cavity proximate the barrel plunger end 1646 and a needle seal 1665 is disposed in the cavity proximate the barrel needle end 1644. The plunger seal 1640 and the needle seal 1665 define a medication space 1688a therebetween in the cavity. The needle seal 1665 includes a sidewall 1665b for engagement with an interior surface the barrel 1642 and includes an top wall 1665a to provide a generally C-shape to the needle seal 1665 when viewed in a side cross-sectional view, FIG. 16A. The plunger seal 1640 and the needle seal 1665 cooperate to retain the medication 1688 in the medication space 1688a, FIG. 16B. A double-ended needle 1652 is attached to the needle end 1644 of the elongated barrel 1642 with the proximal tip 1657 of the double-ended needle 1652 disposed in the cavity between the needle seal 1665 and the needle end 1644 of the barrel 1642. In this configuration the needle 1652 is isolated from the medication 1688 and the medication space 1688a. In operation the plunger seal 1640 slides within the cavity along the longitudinal axis X-X and movement of the plunger seal 1640 towards the needle end 1644 of the barrel 1642 causes the medication 1688 to move towards the double-ended needle 1652. Movement of the medication 1688 causes the needle seal 1665 to move into contact with the proximal tip 1657 of the double-ended needle 1652 to pierce the needle seal 1665 placing the double-ended needle 1652 in fluid communication with the medication 1688, FIG. 16C.

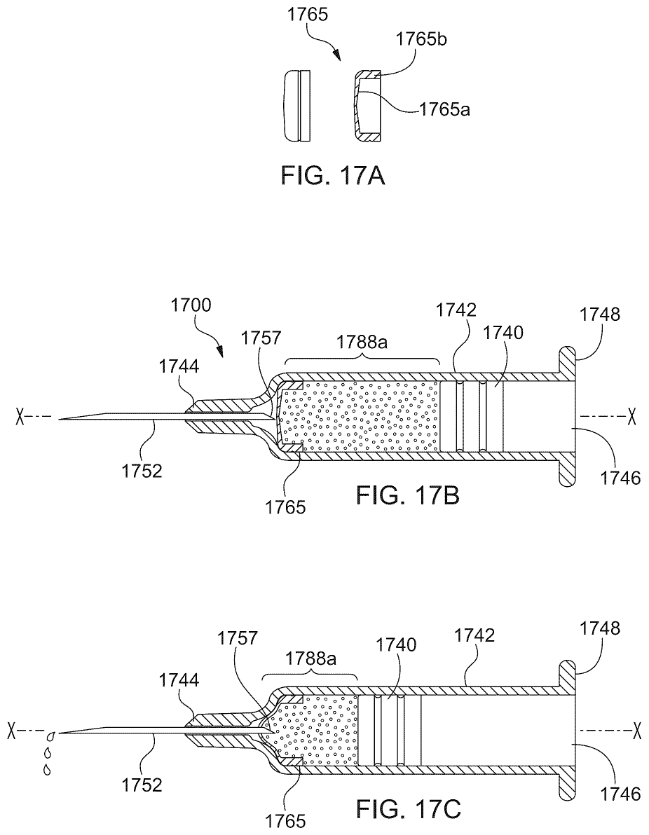

A further exemplary embodiment of a medication syringe 1700 with needle isolated from the medication prior to use is illustrated in FIGS. 17A-17C. The syringe 1700 has an elongated barrel 1742 having a barrel plunger end 1746 and an opposing barrel needle end 1744 and a longitudinal axis X-X extending therebetween. A flange 1748 is provided at the barrel plunger end 1746. The barrel 1742 has a cavity therein for holding a medication 1788. A plunger seal 1740 is disposed in the cavity proximate the barrel plunger end 1746 and a needle seal 1765 is disposed in the cavity proximate the barrel needle end 1744. The plunger seal 1740 and the needle seal 1765 define a medication space 1788a therebetween in the cavity. The needle seal 1765 includes a sidewall 1765b for engagement with an interior surface the barrel 1742 and includes an top wall 1765a to provide a generally C-shape to the needle seal 1765 when viewed in a side cross-sectional view, FIG. 17A. The plunger seal 1740 and the needle seal 1765 cooperate to retain the medication 1788 in the medication space 1788a, FIG. 17B. A double-ended needle 1752 is attached to the needle end 1744 of the elongated barrel 1742 with the proximal tip 1757 of the double-ended needle 1752 disposed in the cavity between the needle seal 1765 and the needle end 1744 of the barrel 1742. In this configuration the needle 1752 is isolated from the medication 1788 and the medication space 1788a. In operation the plunger seal 1740 slides within the cavity along the longitudinal axis X-X and movement of the plunger seal 1740 towards the needle end 1744 of the barrel 1742 causes the medication 1788 to move towards the double-ended needle 1752. Movement of the medication 1788 causes the needle seal 1765, e.g., the top wall 1765a, to expand into contact with the proximal tip 1757 of the double-ended needle 1752 to pierce the needle seal 1765 placing the double-ended needle 1752 in fluid communication with the medication 1788, FIG. 17C.

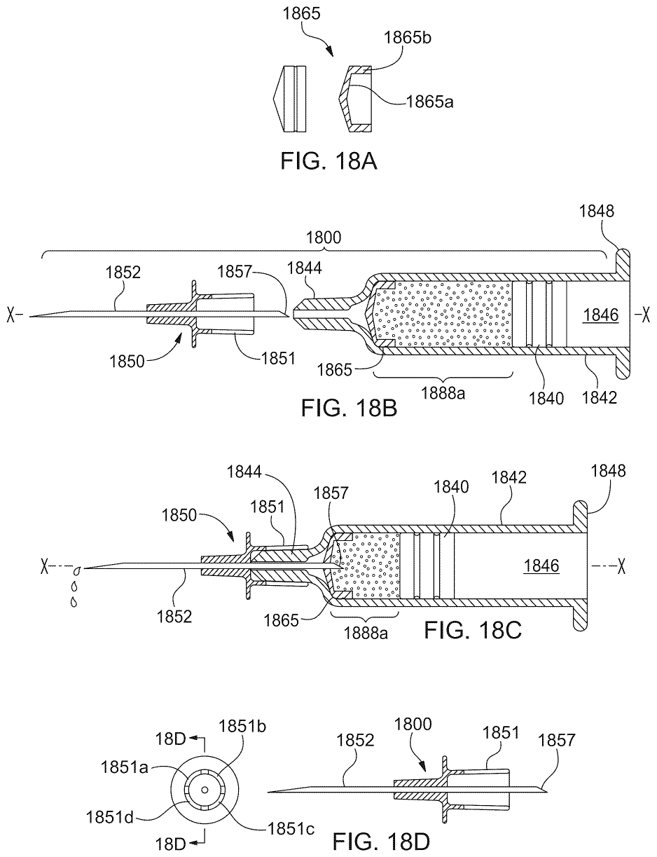

A yet further exemplary embodiment of a medication syringe 1800 with needle isolated from the medication prior to use is illustrated in FIGS. 18A-18D. The syringe 1800 has an elongated barrel 1842 having a barrel plunger end 1846 and an opposing barrel needle end 1844 and a longitudinal axis X-X extending therebetween. A flange 1848 is provided at the barrel plunger end 1846. The barrel 1842 has a cavity therein for holding a medication 1888. A plunger seal 1840 is slidably disposed in the cavity proximate the barrel plunger end 1846 and a needle seal 1865 is disposed in the cavity proximate the barrel needle end 1844. The plunger seal 1840 and the needle seal 1865 define a medication space 1888a therebetween in the cavity. The needle seal 1865 includes a sidewall 1865b for engagement with an interior surface the barrel 1842 and includes an top wall 1865a to provide a generally C-shape to the needle seal 1865 when viewed in a side cross-sectional view, FIG. 18A. The plunger seal 1840 and the needle seal 1865 cooperate to retain the medication 1888 in the medication space 1888a, FIG. 18B. A double-ended needle 1852 is attached to a needle hub 1850. The needle hub 1850 is attachable to the barrel needle end 1844 so that upon placement of the needle hub 1850 on the barrel needle end 1844 the medication seal 1865 is pierced by a proximal tip 1857 of the double-ended needle 1852 to place the double-ended needle 1852 in fluid communication with the cavity to receive medication 1888 therefrom, FIG. 18C. The needle hub 1850 includes a sidewall 1851 having a shape that is complementary to a shape of the barrel needle end 1844. The sidewall 1851 includes a plurality of longitudinal segments 1851a-d that extend along the direction of the longitudinal axis X-X, FIG. 18D. The longitudinal segments 1851a-d are configured to expand in an outward direction from the longitudinal axis X-X to exert a gripping force on the barrel needle end 1844 when the sidewall 1851 is in contact therewith.

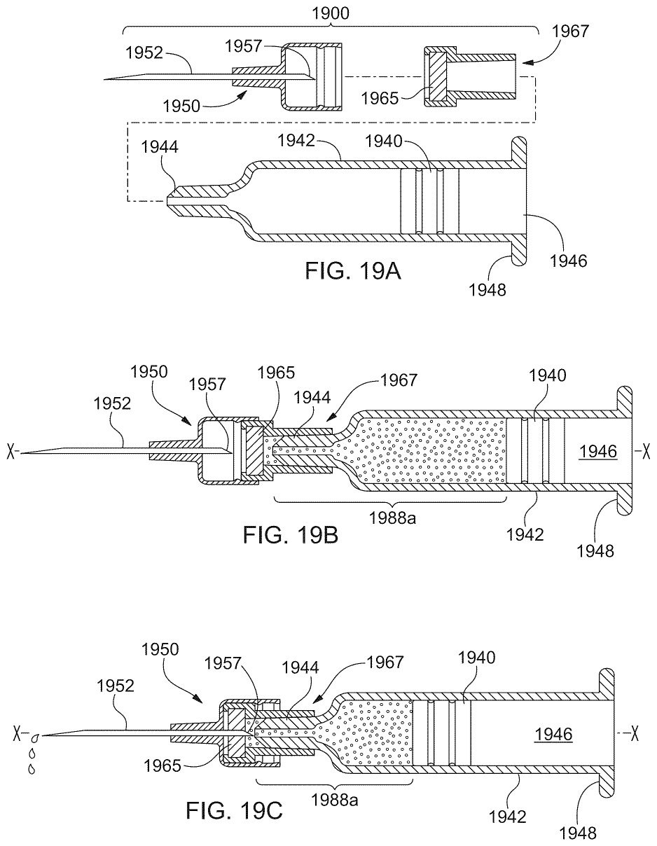

In still yet a further exemplary embodiment of a medication syringe 1900 with needle isolated from the medication prior to use is illustrated in FIGS. 19A-19C. The syringe 1900 has an elongated barrel 1942 having a barrel plunger end 1946 and an opposing barrel needle end 1944 and a longitudinal axis X-X extending therebetween. A flange 1948 is provided at the barrel plunger end 1946. The barrel 1942 has a cavity therein for holding a medication 1988. A plunger seal 1940 is slidably disposed in the cavity proximate the barrel plunger end 1946. A medication plug 1967 has a medication seal 1965 and is attached to the barrel needle end 1944. The medication seal 1965 is in contact with the barrel needle end 1944 to prevent the egress of medication from the cavity through the barrel needle end 1944, FIG. 19B. The plunger seal 1940 and the medication seal 1965 cooperate to retain the medication 1988 in the medication space 1988a. A double-ended needle 1952 is attached to a needle hub 1950. The needle hub 1950 is attachable to the medication plug 1967 so that upon placement of the needle hub 1950 on the medication plug 1967 the medication seal 1965 is pierced by a proximal tip 1957 of the double-ended needle 1952 to place the double-ended needle 1952 in fluid communication with the cavity to receive medication 1988 therefrom, FIG. 19C. The medication plug 1967 may be formed from a rigid material.

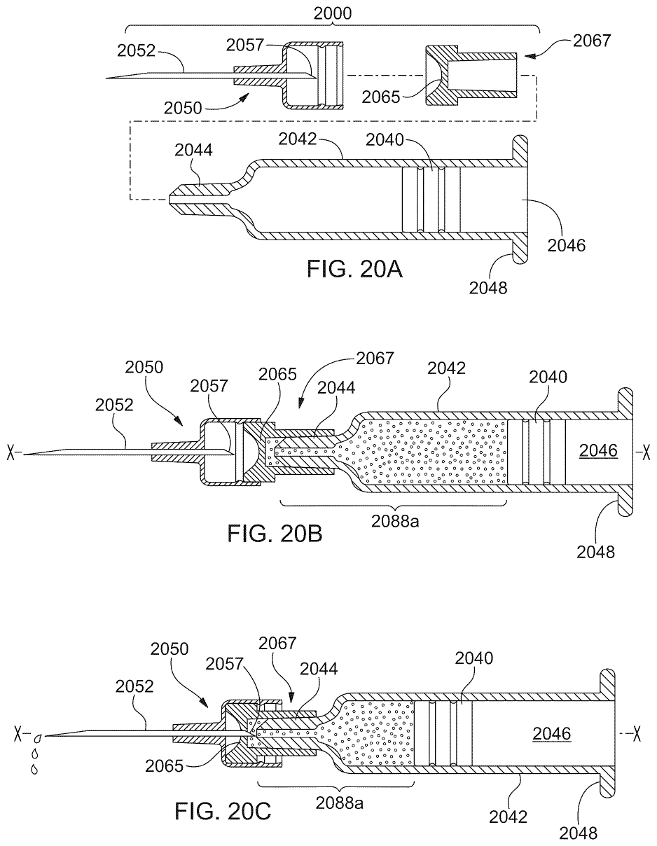

A further exemplary embodiment of a medication syringe 20000 with needle isolated from the medication prior to use is illustrated in FIGS. 20A-20C and is similar in certain respects to the syringe 1900. A notable difference is that the medication plug 2067 may be formed from an elastic material, in which case the medication seal 2065 may be formed from the same material as the medication plug 2067 and the medication seal 2065 and the medication plug 2067 formed as a single unitary part, FIG. 20A. In other respects the syringe 2000 has an elongated barrel 2042 having a barrel plunger end 2046 and an opposing barrel needle end 2044 and a longitudinal axis X-X extending therebetween. A flange 2048 is provided at the barrel plunger end 2046. The barrel 2042 has a cavity therein for holding a medication 2088. A plunger seal 2040 is slidably disposed in the cavity proximate the barrel plunger end 2046. The medication plug 2067 has an integral medication seal 2065 and is attached to the barrel needle end 2044 with the medication seal 2065 in contact with the barrel needle end 2044 to prevent the egress of medication from the cavity through the barrel needle end 2044, FIG. 20B. The plunger seal 2040 and the medication seal 2065 cooperate to retain the medication 2088 in the medication space 2088a. A double-ended needle 2052 is attached to a needle hub 2050. The needle hub 2050 is attachable to the medication plug 2067 so that upon placement of the needle hub 2050 on the medication plug 2067 the medication seal 2065 is pierced by a proximal tip 2057 of the double-ended needle 2052 to place the double-ended needle 2052 in fluid communication with the cavity to receive medication 2088 therefrom, FIG. 20C.

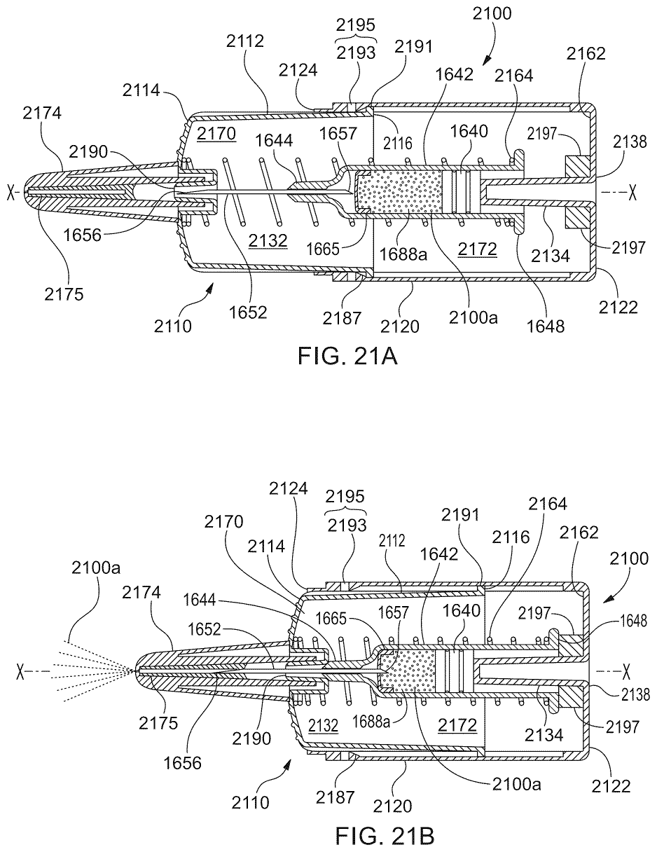

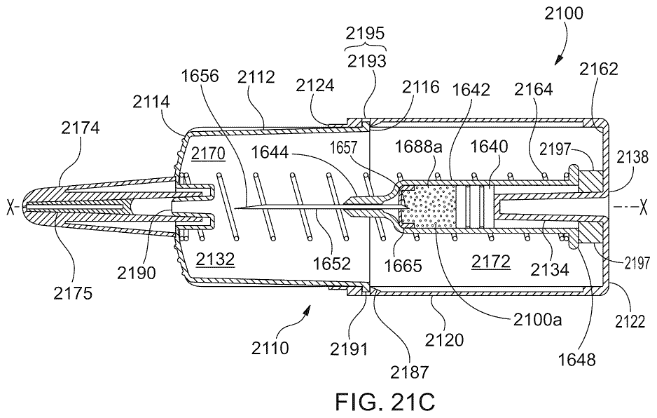

The present disclosure also provides, in another of its aspects, a multimode nasal medication injector housing by itself, for use with a needle-based injection system (NIS), but not including the NIS, FIGS. 21A-25C. (In fact, any housing disclosed herein may be provided without an NIS, if desired, such as when an latter party supplies the NIS.) In this regard, a multimode nasal medication injector housing 2100, 2200, 2300, 2400, 2500 capable of conversion from a nasal modality of medication delivery to an intravenous delivery mode and an intramuscular medication delivery mode is provided. A front housing 2112, 2212, 2312, 2412, 2512 having a front housing distal end 2114, 2214, 2314, 2414, 2514 and having a front housing proximal end 2116, 2216, 2316, 2416, 2516 is provided. A rear housing 2120, 2220, 2320, 2420, 2520 having a rear housing distal end 2124, 2224, 2324, 2424, 2524 and having a rear housing proximal end 2122, 2222, 2322, 2422, 2522 is also provided. The front housing proximal end 2116-2516 is slidably coupled to the rear housing distal end 2124-2524 to define a housing cavity therein and to permit the front and rear housings 2112-2512, 2120-2520 to translate relative to one another along a longitudinal axis X-X extending therethrough. A plunger 2134, 2234, 2334, 2434, 2534 is attached the interior to the rear housing 2120-2520 at a selected location at the rear housing proximal end 2122-2522. The plunger 2134-2534 may be permanently attached and extends along the longitudinal axis X-X and has opposing distal and proximal plunger ends; the proximal plunger end is in contact with the rear housing 2120-2520. The plunger may be provided is an integral part of the rear housing 2120-2520 so the plunger 2134-2534 and rear housing 2120-2520 are seamlessly connected as a unitary whole. A medication spacer 2197, 2297, 2397, 2497, 2597 having a thickness extending along the direction of the longitudinal axis X-X is disposed internal to the housing cavity at the selected location at the rear housing proximal end 2122-2522. The medication spacer 2197-2597 may have an annular shape with a hole therein and is disposed around the plunger 2134-2534 with the plunger 2134-2534 in the hole.

A nasal nozzle 2174, 2274, 2374, 2574 is provided having distal and proximal nozzle ends with a canal disposed interior thereto between the distal and proximal nozzle ends. The nasal nozzle 2174-2574 extends along the longitudinal axis X-X, and the proximal nozzle end is removably coupled to the front housing 2112-2512 at the front housing distal end. The nasal nozzle 2174-2574 has one or more openings at the distal nozzle end through which the medication is delivered from the multimode nasal medication injector housing 2100-2500. A canal seal 2175, 2275, 2375, 2475, 2575 may be provided in the canal of the nasal nozzle 2174-2574.

Lockout structures may also be provided in the same form as those shown and described in connection with FIGS. 9A-9C. For example, lockout structures may include a tab 2191, 2291, 2391, 2491, 2591 which is disposed on a selected one of the front housing 2112-2512 or rear housing 2120-2520 and may have a complementary slot 2193, 2293, 2393, 2493, 2593 disposed on the other one of the front housing 2112-2512 and rear housing 2120-2520. The slot 2193-2593 is dimensioned to receive and hold the tab 2191-2591. The tab 2191-2591 may be disposed on a proximal end 2116-2516 of the front housing 2112-2512 and the slot 2193-2593 disposed on a distal end 2124-2524 of the rear housing 2120-2520. The rear housing 2120-2520 may include a channel 2189, 2289, 2389, 2489, 2589 in which the tab 2191-2591 can travel, upon relative movement between the front and rear housings 2112-2512, 2120-2520. The tab 2191-2591 and slot 2193-2593 cooperate to lock the front and rear housings 2112-2512, 2120-2520 when the tab 2191-2591 is in the slot 2193-2593 to prevent movement between the front housing 2112-2512 and rear housing 2120-2520.

In addition, the housings 2100-2500, may also be provided with a needle-based injection system (NIS) 2100a, 2200a, 2300a, 2400a, 2500a disposed in the housing cavity, FIGS. 21A-25C. The NIS 2100a-2500a contains the medication 1688, 1788, 1888, 1988, 2088 to be delivered, and has opposing proximal and distal NIS ends and is disposed along the longitudinal axis X-X. The NIS 2100a-2500a has a plunger seal 1640, 1740, 1840, 1940, 2040 slidably disposed therein. The plunger seal 1640-2040 is operably connected to the distal plunger end of the housing 2000-2025. The NIS 2100a-2500a has a needle 1652, 1752, 1852, 1952, 2052 disposed along the longitudinal axis X-X between the distal NIS end and the front housing distal end 2114-2514.

The housing 2100-2500 includes a biasing member, such as a spring 2164, 2264, 2364, 2464, 2564. The spring 2164-2564 may be positioned in the housing cavity along the longitudinal axis X-X between the front and rear housings 2112-2512, 2120-2520. The spring 2164-2564 may be disposed around the plunger 2134-2534, and the spring 2164-2564 may have a proximal end positioned against a spring abutment surface, such as the syringe flange 1648, 1748, 1848, 1948, 2048. The spring 2164-2564 is configured to exert force outwardly to keep the front housing 2112-2512 and the rear housing 2120-2520 positioned in a first position prior to use, FIGS. 21A, 22A, 23A, 24A, 25A. Desirably, the needle 2152-2552 is not in fluid communication with the medication 1688-2088 when the front and rear housings 2112-2512, 2120-2520 are in the first position, FIGS. 21A-25A.

The spring 2164-2564 is compressible upon application of force along the longitudinal axis X-X to urge the front housing distal end 2114-2514 towards the rear housing proximal end 2122-2522 so that the front and rear housings 2112-2512, 2120-2520 are positioned at a second position, FIGS. 21B, 22B, 23B, 24B, 25B. A proximal end 1657, 1757, 1857, 1957, 2057 of the double-ended needle 2152-2552 is configured to pierce a seal on the NIS 2100a-2500a upon movement of the front and rear housings 2112-2512, 2120-2520 towards one another thereby placing the double-ended needle 2152-2552 in fluid communication with the medication 1688-2088 in the NIS 2100a-2500a. Upon movement of the injector housing from the first position to the second position, the plunger seal 1640-2040 pushes the medication 1688-2088 out of the NIS 2100a-2500a, through the needle 2152-2552, and out of a distal end of the needle 2152-2552 for delivery to a patient. When the canal seal 2175-2575 is pierced by the distal needle tip 1656, 1756, 1856, 1956, 2056, the canal seal 2175-2575 provides a fluid-tight seal around the needle 1652-2052. The needle 1652-2052 extends outward beyond the front housing distal end 2114-2514 a selected length.

The spring 2164-2564 may have elastic potential energy stored therein when compressed in the second position. The elastic potential energy is sufficient to move the front and rear housings 2112-2512, 2120-2520 away from each other in a direction along the longitudinal axis X-X to a third position upon release of the elastic potential energy, FIGS. 21C, 22C, 23C, 24C, 25C. In the third position the needle 2152-2552 is fully enclosed in the housing cavity of the front and rear housings 2112-2512, 2120-2520. The nasal nozzle 2174-2574 may have a length greater than the selected length to contain the needle 2152-2552 in the canal when the injector is in the second position.

The present disclosure also provides a method of assembling a multimode nasal medication injector capable of conversion from a nasal modality of medication delivery to an intravenous delivery mode and an intramuscular medication delivery mode. The method comprises providing a multimode nasal medication injector housing of the present disclosure; inserting a needle-based injection system (NIS) containing the medication to be delivered in the housing cavity along the longitudinal axis, the needle-based injection system having a plunger seal slidably disposed therein; operably connecting the distal plunger end of the plunger to the plunger seal of the NIS; placing a spring in the housing cavity with a proximal end of the spring in contact with a distal spring abutment surface, the spring configured to exert force outwardly to keep the front housing and the rear housing positioned in a first position prior to use, the spring being compressible upon application of force along the longitudinal axis to urge the front housing distal end towards the rear housing proximal end so that the front and rear housings are positioned at a second position and such that the needle is disposed in fluid communication with the medication in the needle-based injection system and the needle extends outward beyond the front housing distal end a selected length; attaching the front housing over the NIS; and fixedly and removably attaching the nasal nozzle to the front housing distal end. The step of inserting a NIS may include providing a medication syringe having an elongated barrel with opposing needle and plunger ends. The medication syringe may include a flange at the barrel plunger end and the spring may be placed around the elongated barrel, and a selected end of the spring may be placed against the flange. The NIS in the method may be any of the exemplary medication syringes of the present disclosure. Alternatively, the NIS in the method may include a medication cartridge and wherein the distal spring abutment surface a selected surface of the rear housing at the rear housing distal end. In a further aspect of the method, the method may include, prior to placing the spring, providing a medication spacer having a thickness that extends along the direction of the longitudinal axis and placing the medication spacer in the housing cavity at the selected location at the rear housing proximal end. The method may also include attaching a removable cap to the front housing distal end to cover the nasal nozzle.

TABLE 1
Selected Reference numerals^{†}

| Ref. | Element | Ref. | Element |
|---|---|---|---|
| 10, 910 | injector | 68, 968 | spring second end |
| 12, 912, | front housing | 70, 970 | front housing interior |
| 2112-2512 | | | |
| 14, 914 | front housing distal end | 72, 972 | rear housing interior |
| 16, 916 | front housing proximal end | 74, 974, | nasal nozzle |
| | | 2174-2574 | |
| 18 | front housing wall | 76 | nasal nozzle canal |
| 20, 920, | rear housing | 78, 978 | nasal nozzle tip |
| 2120-2520 | | | |
| 22, 922 | rear housing proximal end | 80, 980 | nozzle linkage |
| 24, 924 | rear housing distal end | 82, 982 | cap coupler |
| 26 | rear housing wall | 84, 984 | injector coupler |
| 28, 928 | cap | 985 | picc line |
| 32, 932, | NIS: syringe assembly, | 86, 986 | aperture |
| 1600-1700, | medication cartridge, syringe | | |
| 2100a-2500a | | | |
| 34, 934, | plunger | 987 | shoulder |
| 2134-2534 | | | |
| 36, 936 | plunger distal end | 88, 988 | barrel interior |
| 38, 938 | plunger proximal end | 989 | channel |
| 40, 940, | plunger seal | 90, 990 | injector lock |
| 1640-2040 | | | |
| 42, 942, | barrel | 92, 992 | injector lock base |
| 1642-2042 | | | |
| 44, 944, | barrel needle end | 94 | label |
| 1644-2044 | | | |
| 46, 1646- | barrel plunger end | 96 | label perforations |
| 2046 | | | |
| 48, 1648- | flange | 991, 2191-2591 | lockout tab |
| 2048 | | | |
| 50, 950, | hub | 993, 2193-2593 | lockout slot |
| 1850-2050 | | | |
| 52, 952, | needle | 995, 2195-2595 | lockout |
| 1652-2052 | | | |
| 54 | needle lumen | 997, 997a-b, | medication spacer |
| | | 2197-2597 | |
| 56, 956, | needle tip, distal needle tip | 2100-2500 | multimode nasal |
| 1656-2056 | | | medication injector |
| | | | housing |
| 957, 1657- | proximal needle tip | 100 | medication |
| 2057 | | | |
| 58 | needle base | 102 | finger |
| 60, 965 | self-sealing membrane | 104 | tissue |
| 62 | plunger stop | 106 | direction of force |
| 64, 964, | spring | X-X | longitudinal axis |
| 2164-2564 | | | |
| 1665-1865 | needle seal | Y | longitudinal marker |
| 66, 966 | spring first end | Z | length |
| 1967, 2067 | medication plug | | |

^{†}Ranges of reference numeral ranges are abbreviated with the last two digits remaining constant while leading digit(s) change in single integer increments, e.g. 2112-2512 means 2112, 2212, 2312, 2412, 2512; 2100-2500 means 2100, 2200, 2300, 2400, 2500, and so forth. This convention is used throughout the disclosure.

As used herein the terms “permanently attached” or “non-removably attached” mean that the parts so attached cannot be separated from one another without damaging or otherwise altering one or both of the parts. As used herein, the terms “removably attached”, “removably coupled”, “removably attachable”, “removably couplable”, or “removable” mean that a first object that is coupled to a second object may be decoupled from the second object, or taken away from a coupled or attached position relative to the second object, using some force or movement, without damaging either object.

These and other advantages of the present disclosure will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

What is claimed is:

1. A medication syringe, comprising:
an elongated barrel having a barrel plunger end and an opposing barrel needle end and a longitudinal axis extending therebetween, the barrel having a cavity therein for holding a medication;
a plunger seal slidably disposed in the cavity proximate the barrel plunger end;
a medication plug having a medication seal disposed therein, the medication plug attached to the barrel needle end of the elongated barrel with the medication seal in contact with the barrel needle end of the elongated barrel to prevent the egress of medication from the cavity through the barrel needle end;
a double-ended needle attached to a needle hub, the needle hub attachable to the medication plug so that upon placement of the needle hub on the medication plug the medication seal is pierced by a selected end of the double-ended needle to place the double-ended needle in fluid communication with the cavity to receive medication therefrom.

2. The medication syringe according to claim 1, wherein the medication plug is formed from a rigid material.

3. The medication syringe according to claim 1, wherein the medication plug is formed from an elastic material.

4. The medication syringe according to claim 3, wherein the medication seal is formed from the same material as the medication plug.

5. The medication syringe according to claim 4, wherein the medication seal and the needle hub are formed as a single unitary part.

6. A multimode nasal medication injector housing for use with a needle-based injection system (NIS), the multimode nasal medication injector housing capable of conversion from a nasal modality of medication delivery to an intravenous delivery mode and an intramuscular medication delivery mode, the multimode nasal medication injector housing comprising:
a front housing having a front housing distal end and a front housing proximal end and a rear housing having a rear housing distal end and a rear housing proximal end, the front housing proximal end slidably coupled to the rear housing distal end to define a housing cavity therein and to permit the front and rear housings to translate relative to one another along a longitudinal axis extending therethrough;
a plunger permanently attached interior to the rear housing at a selected location at the rear housing proximal end, the plunger extending along the longitudinal axis and a having opposing distal and proximal plunger ends, the proximal plunger end in contact with the rear housing;
a nasal nozzle having distal and proximal nozzle ends with a canal disposed interior thereto between the distal and proximal nozzle ends, the nasal nozzle extending along the longitudinal axis, the proximal nozzle end removably coupled to the front housing at the front housing distal end, the nasal nozzle having one or more openings at the distal nozzle end through which the medication is delivered from the multimode nasal medication injector housing.

7. The multimode nasal medication injector housing according to claim 6, wherein the plunger is an integral part of the rear housing so the plunger and rear housing are seamlessly connected as a unitary whole.

8. The multimode nasal medication injector housing according to claim 6, comprising a medication spacer having a thickness extending along the direction of the longitudinal axis, the medication spacer disposed internal to the housing cavity at the selected location at the rear housing proximal end.

9. The multimode nasal medication injector housing according to claim 8, wherein the medication spacer has an annular shape with a hole therein and is disposed around the plunger with the plunger in the hole.

10. The multimode nasal medication injector housing according to claim 6, wherein the plunger is configured to be attached to a plunger seal in an NIS.

11. The multimode nasal medication injector according to claim 6, comprising a lockout structure comprising a tab disposed on a selected one of the first and second housings and a complementary slot disposed on the other one of the first and second housings, the slot dimensioned to receive and hold the tab.

12. The multimode nasal medication injector according to claim 11, wherein the tab is disposed on a proximal end of the front housing and the slot is disposed on a distal end of the rear housing and the rear housing includes a channel in which the tab can travel upon relative movement between the front and rear housings.

13. The multimode nasal medication injector housing according to claim 11, wherein the tab and slot cooperate to lock the front and rear housings at a relative location that encloses the needle entirely within the injector when the tab is in the slot to prevent movement between the front and rear housings.

14. The multimode nasal medication injector housing according to claim 6, comprising:
a NIS containing the medication to be delivered, the NIS disposed in the housing cavity, the NIS having opposing proximal and distal NIS ends and disposed along the longitudinal axis, the NIS having a plunger seal slidably disposed therein operably connected to the distal plunger end, and having a needle disposed along the longitudinal axis between the distal NIS end and the front housing distal end; and
a spring positioned in the housing cavity along the longitudinal axis between the front and rear housings, the spring configured to exert force outwardly to keep the front housing and the rear housing positioned in a first position prior to use, the spring being compressible upon application of force along the longitudinal axis to urge the front housing distal end towards the rear housing proximal end so that the front and rear housings are positioned at a second position and such that the needle is disposed in fluid communication with the medication in the NIS and the needle extends outward beyond the front housing distal end a selected length.

15. The multimode nasal medication injector housing according to claim 14, wherein the spring has elastic potential energy when compressed in the second position, the elastic potential energy sufficient to move the front and rear housings away from each other in a direction along the longitudinal axis to a third position upon release of the elastic potential energy with the needle fully enclosed in the housing cavity of the front and rear housings when disposed in the third position.

16. The multimode nasal medication injector housing according to claim 14, wherein the needle is in fluid communication with the medication in the NIS when the front and rear housings are in the first position.

17. The multimode nasal medication injector housing according to claim 14, wherein the needle is not in fluid communication with the medication when the front and rear housings are in the first position.

18. The multimode nasal medication injector housing according to claim 17, wherein the needle is in fluid communication with the medication in the NIS when the front and rear housings are in the second position.

19. The multimode nasal medication injector housing according to claim 14, wherein when the injector housing is moved from the first position to the second position, the plunger seal pushes the medication out of the NIS, through the needle, and out of a distal end of the needle for delivery to a patient.

20. The multimode nasal medication injector housing according to claim 14, wherein a proximal end of the double-ended needle is configured to pierce a seal on the NIS upon movement of the front and rear housings towards one another thereby placing the double-ended needle in fluid communication with the medication in the NIS.

21. The multimode nasal medication injector housing according to claim 14, wherein the nasal nozzle has a length greater than the selected length to contain the needle in the canal when the injector is in the second position.

22. The multimode nasal medication injector housing according to claim 14, wherein the NIS includes a medication cartridge containing the medication to be delivered, the medication cartridge disposed in the housing cavity, the cartridge having opposing proximal and distal ends and disposed along the longitudinal axis, the cartridge containing the plunger seal slidably disposed therein.

23. The multimode nasal medication injector housing according to claim 22, wherein the spring is disposed around the plunger.

24. The multimode nasal medication injector housing according to claim 22, wherein the spring has two opposing ends and a selected end is disposed against the rear housing distal end.

25. The multimode nasal medication injector housing according to claim 14, wherein the NIS comprises a medication syringe, the medication syringe comprising:
an elongated barrel having a barrel plunger end and an opposing barrel needle end and a longitudinal axis extending therebetween, the barrel having a cavity therein for holding a medication;
a plunger seal slidably disposed in the cavity proximate the barrel plunger end;
a medication plug having a medication seal disposed therein, the medication plug attached to the barrel needle end of the elongated barrel with the medication seal in contact with the barrel needle end of the elongated barrel to prevent the egress of medication from the cavity through the barrel needle end;
a double-ended needle attached to a needle hub, the needle hub attachable to the medication plug so that upon placement of the needle hub on the medication plug the medication seal is pierced by a selected end of the double-ended needle to place the double-ended needle in fluid communication with the cavity to receive medication therefrom.

26. The multimode nasal medication injector housing according to claim 25, wherein the spring is disposed around the elongated barrel.

27. The multimode nasal medication injector housing according to 26, wherein the medication syringe includes a flange at the barrel plunger end and the spring has two opposing ends and a selected end is disposed against the flange.
